(19) United States
(12) Patent Application Publication
(10) Pub. No.: US 2025/0388550 A1
(43) Pub. Date: Dec. 25, 2025

(54) SALTS OF AN ANTIOXIDANT AND CRYSTALLINE FORMS THEREOF

(71) Applicants: Jan Trampota, Fairfield, NJ (US); Mironova Labs, Inc., Fairfield, NJ (US)

(72) Inventors: Jan Trampota, Fairfield, NJ (US); Stephan D. Parent, West Lafayette, IN (US); Travis Lee Houston, West Lafayette, IN (US)

(21) Appl. No.: 18/880,330

(22) PCT Filed: Jun. 30, 2023
(86) PCT No.: PCT/US23/26809

§ 371 Date: Dec. 31, 2024

Related U.S. Application Data
(60) Provisional application No. 63/357,804, filed on Jul. 1, 2022.

(51) Int. Cl. C07D 233/84; A61K 31/4172; C01B 7/01; C01B 17/69; C01B 25/18; C07C 53/10; C07C 57/15; C07C 59/255; C07C 63/08

(52) U.S. Cl. CPC C07D 233/84; A61K 31/4172; C01B 7/01; C01B 17/69; C01B 25/18; C07C 53/10; C07C 57/15; C07C 59/255; C07C 63/08; C01P 2002/72; C07B 2200/13

(57) ABSTRACT

The present disclosure is directed to salt forms of L-Ergothioneine and crystalline forms thereof, and processes for their preparation. Also provided is a crystalline form of L#Ergothioneine (Form C) and a crystalline form of DL-Ergothioneine (Form A).

### CROSS-REFERENCE TO RELATED APPLICATIONS

This international applications claims the benefit of U.S. Provisional Application No. 63/357,804 filed Jul. 1, 2023, the entirety of which is incorporated by reference for all purposes.

### FIELD

The present disclosure is directed to salt forms of L-Ergothioneine and crystalline forms thereof, and processes for their preparation. Also provided is a crystalline form of L-Ergothioneine anhydrate (Form C) and a crystalline form of DL-Ergothioneine (Form A).

### BACKGROUND

L-Ergothioneine is a naturally occurring thio-histidine betaine amino acid. The compound is only synthesized by certain microbes, including bacteria Actinomycetota and Cyanobacteria, and certain fungi. Human and animals must obtain L-Ergothioneine exclusively through diet, for example in kidney beans, black beans, oat bran, and mushrooms.

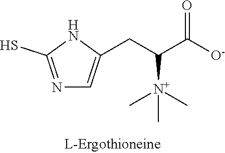

Although humans do not produce L-Ergothioneine, it is reported to have antioxidant activity and cytoprotective properties. In fact, humans have evolved a selective transporter for L-Ergothioneine, solute carrier family 22, member 4 (SLC22A4). Once consumed, L-Ergothioneine accumulates in tissues high in SLC22A4, including erythrocytes, bone marrow, the liver, the kidney, and eyes.

There are multiple lines of evidence that L-Ergothioneine acts as a potent antioxidant and cellular protectant (Borodina et al. Nutr Res Rev 2020, 33:190). For example, L-Ergothioneine has been shown to decrease oxidative stress levels in the liver and kidneys of rats (Deiana et al. Clin. Nutr., 2004, 23:183). It has also been studied as an antioxidant in certain disease states, including acute respiratory distress (Repine et al. Prev Med 2012, 54: S79) and chronic obstructive pulmonary disease (COPD) (Rahman, Biochim Biophys Acta, 2012, 1822:714). Its antioxidant properties also make it common ingredient in cosmetics for protection against oxidative stress.

Typically, L-Ergothioneine is produced through biological extraction or chemical synthesis. A crystal form of L-Ergothioneine dihydrate was first reported in Sugihara et al. (Acta Cryst, 1976, B32: 181). In 2005, an anhydrate crystalline form of L-Ergothioneine was also reported (Hand et al. J. Nat. Prod. 2005, 68:293).

Given the importance of L-Ergothioneine in the fields of food, medicine, and cosmetics, it would be beneficial to develop highly purified and stable forms of L-Ergothioneine that can be easily manufactured and formulated. There is also a need for salts and crystalline forms of L-Ergothioneine that can act as and provide these advantageous forms.

### SUMMARY

In one embodiment, the present disclosure provides salt forms of L-Ergothioneine and crystalline forms thereof.

In one embodiment, the salt form of L-Ergothioneine is L-Ergothioneine phosphoric acid.

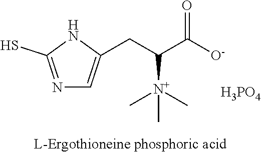

In one embodiment, the L-Ergothioneine phosphoric acid is crystalline. In one embodiment, the L-Ergothioneine phosphoric acid is the co-crystal L-Ergothioneine phosphoric acid (Form Λ). It cannot be predicted in advance whether a compound exists in one or more solid forms, or whether the properties of the solid form are advantageous for manufacturing and pharmaceutical formulation. In particular, it has been discovered that the co-crystal L-Ergothioneine phosphoric acid (Form A) is easy to manufacture due to its low solubility in polar protic solvents. This is useful for synthesis and purification purposes of both L-Ergothioneine phosphoric acid (Form A) and L-Ergothioneine.

In alternative embodiments, provided herein are crystalline forms selected from L-Ergothioneine acetate (Form A), L-Ergothioneine benzoate (Form A), L-Ergothioneine hemifumurate (Form A), L-Ergothioneine hydrochloride (Form A), L-Ergothioneine sulfate (Form A), and L-Ergothioneine hemitartrate (Form A).

In another aspect, provided herein is a crystalline form of L-Ergothioneine, anhydrate free base L-Ergothioneine (Form C).

In another aspect, provided herein is a crystalline form of DL-Ergothioneine (Form A).

In another aspect, provided herein are pharmaceutical compositions comprising a crystalline form of L-Ergothioneine described herein, including the co-crystal or salt forms of L-Ergothioneine. In one embodiment, the pharmaceutical composition comprises co-crystal L-Ergothioneine phosphoric acid (Form A). In one embodiment, the pharmaceutical comprises L-Ergothioneine prepared with L-Ergothioneine phosphoric acid.

Also provided herein are pharmaceutical compositions comprising the crystalline form of DL-Ergothioneine (Form A) and pharmaceutical compositions prepared with DL-Ergothioneine (Form A).

Also provided herein is a method for treating diseases or disorders caused by oxidative stress and/or inflammation comprising administering a crystalline form of L-Ergothioneine described herein, including the co-crystal or salt forms of L-Ergothioneine, the crystalline form of DL-Ergothioneine (Form A), or a pharmaceutical composition thereof to a patient in need thereof.

Alternative salt forms of L-Ergothioneine provided herein are selected from the group consisting of L-Ergothioneine acetate, L-Ergothioneine benzoate, L-Ergothioneine hemifumurate, L-Ergothioneine phosphoric acid, L-Ergothioneine sulfate, L-Ergothioneine hemitartrate, L-Ergothioneine hemimaleate, L-Ergothioneine maleate, L-Ergothioneine adipic acid, L-Ergothioneine L-aspartic acid, L-Ergothioneine citric acid, L-Ergothioneine camphorsulfonic acid, L-Ergothioneine methanesulfonic sulfonic acid, and L-Ergothioneine tosylate. In one embodiment, provided herein is a salt form of L-Ergothioneine selected from the group consisting of L-Ergothioneine acetate, L-Ergothioneine benzoate, L-Ergothioneine hemifumurate, L-Ergothioneine phosphoric acid, L-Ergothioneine sulfate, and L-Ergothioneine hemitartrate. In one embodiment, provided herein is a salt form of L-Ergothioneine selected from the group consisting of L-Ergothioneine hemifumurate, L-Ergothioneine hemitartrate, L-Ergothioneine hemimaleate, L-Ergothioneine adipic acid, L-Ergothioneine L-aspartic acid, and L-Ergothioneine citric acid. In one embodiment, provided herein is L-Ergothioneine phosphoric acid.

Also provided herein are a pharmaceutical compositions comprising a salt form of L-Ergothioneine described herein and methods for the treatment of diseases or disorders caused by oxidative stress and/or inflammation comprising administering a salt form of L-Ergothioneine described herein.

### BRIEF DESCRIPTION OF FIGURES

FIG. 1 is an atomic displacement ellipsoid diagram of L-Ergothioneine phosphoric acid co-crystal Form A.

FIG. 2 is the XRPD pattern of L-Ergothioneine phosphoric acid co-crystal Form A.

FIG. 3 is the TGA graph of L-Ergothioneine phosphoric acid co-crystal Form A.

FIG. 4 is the DSC graph of L-Ergothioneine phosphoric acid co-crystal Form A.

FIG. 5 is the XRPD pattern of L-Ergothioneine acetate Form A.

FIG. 6 is the TGA graph of L-Ergothioneine acetate Form A.

FIG. 7 is the DSC graph of L-Ergothioneine acetate Form A.

FIG. 8 is the XRPD pattern of L-Ergothioneine benzoate Form A.

FIG. 9 is the TGA graph of L-Ergothioneine benzoate Form A.

FIG. 10 is the DSC graph of L-Ergothioneine benzoate Form A.

FIG. 11 is the XRPD pattern of L-Ergothioneine calcium chloride Material A.

FIG. 12 is the XRPD pattern of L-Ergothioneine hemifumarate Form A.

FIG. 13 is the TGA graph of L-Ergothioneine hemifumarate Form A.

FIG. 14 is the DSC graph of L-Ergothioneine hemifumarate Form A.

FIG. 15 is the XRPD pattern of L-Ergothioneine hydrochloride Form A.

FIG. 16 is the TGA graph of L-Ergothioneine hydrochloride Form A.

FIG. 17 is the DSC graph of L-Ergothioneine hydrochloride Form A.

FIG. 18 is the XRPD pattern of L-Ergothioneine sulfate Form A.

FIG. 19 is the TGA graph of L-Ergothioneine sulfate Form A.

FIG. 20 is the DSC graph of L-Ergothioneine sulfate Form A.

FIG. 21 is the XRPD pattern of L-Ergothioneine hemitartrate Form A.

FIG. 22 is the TGA graph of L-Ergothioneine hemitartrate Form A.

FIG. 23 is the DSC graph of L-Ergothioneine hemitartrate Form A.

FIG. 24 is an atomic displacement ellipsoid diagram of L-Ergothioneine Form C anhydrate.

FIG. 25 is the XRPD pattern of L-Ergothioneine Form C anhydrate.

FIG. 26 are XRPD patterns of L-Ergothioneine Form A anhydrate, L-Ergothioneine Form B dihydrate, and L-Ergothioneine Form C anhydrate.

FIG. 27 is the TGA graph of L-Ergothioneine Form C anhydrate.

FIG. 28 is the DSC graph of L-Ergothioneine Form C anhydrate.

FIG. 29 is the DVS isotherm of L-Ergothioneine Form C anhydrate.

FIG. 30 is the XRPD pattern of DL-Ergothioneine Form A.

### DETAILED DESCRIPTION

The present disclosure provides salts of L-Ergothioneine and crystalline forms thereof. In a preferred embodiment, the salt form of L-Ergothioneine is L-Ergothioneine phosphoric acid. In one embodiment, the L-Ergothioneine phosphoric acid is crystalline. In one embodiment, the L-Ergothioneine phosphoric acid is the co-crystal L-Ergothioneine phosphoric acid (Form A).

The present disclosure also provides a crystalline form of L-Ergothioneine, anhydrate free base L-Ergothioneine (Form C).

The present disclosure also provides a crystalline form of DL-Ergothioneine (Form A).

The present disclosure provides at least the following embodiments:
a) L-Ergothioneine phosphoric acid;
b) Co-crystal L-Ergothioneine phosphoric acid (Form A);
c) A pharmaceutical composition comprising (a) or (b);
d) A pharmaceutical composition comprising L-Ergothioneine prepared from L-Ergothioneine phosphoric acid;
e) The pharmaceutical composition of (d) in a dosage form suitable for topical administration;
f) A method to treat a disease or disorder caused by oxidative stress and/or inflammation comprising administering to a patient in need thereof any one of (a)-(e);
g) An L-Ergothioneine crystalline form selected from L-Ergothioneine acetate (Form A), L-Ergothioneine benzoate (Form A), L-Ergothioneine hemifumurate (Form A), L-Ergothioneine hydrochloride (Form A), L-Ergothioneine sulfate (Form A), and L-Ergothioneine hemitartrate (Form A);
h) Crystalline anhydrate free base L-Ergothioneine (Form C);
i) Crystalline DL-Ergothioneine (Form A);
j) A salt form of L-Ergothioneine selected from the group consisting of L-Ergothioneine acetate, L-Ergothioneine benzoate, L-Ergothioneine hemifumurate, L-Ergothioneine phosphoric acid, L-Ergothioneine sulfate, L-Ergothioneine hemitartrate, L-Ergothioneine hemimaleate, L-Ergothioneine maleate, L-Ergothioneine adipic acid, L-Ergothioneine L-aspartic acid, L-Ergothioneine citric acid, L-Ergothioneine camphorsulfonic acid, L-Ergothioneine methanesulfonic sulfonic acid, and L-Ergothioneine tosylate;
k) A pharmaceutical composition comprising any one of (g)-(j); and
l) A method to treat a disease or disorder caused by oxidative stress and/or inflammation comprising administering any one of (g)-(k) in a patient in need thereof.

### Definitions

Unless otherwise defined, all terms of art, notations and other scientific terminology used herein are intended to have the meanings commonly understood by those of skill in the art to which this invention pertains. In some cases, terms with commonly understood meanings are defined herein for clarity and/or for ready reference, and the inclusion of such definitions herein should not necessarily be construed to represent a difference over what is generally understood in the art. The techniques and procedures known in the art that are described or referenced herein are generally well understood and commonly employed using conventional methodologies by those skilled in the art.

As used herein, the singular forms “a,” “an,” and “the” include the plural referents unless the context clearly indicates otherwise.

As used herein, the term “about” refers to the stated value plus or minus 10%, plus or minus 5%, or plus or minus 1%. For example, a value of “about 10” can encompass a range of 9 to 11. For logarithmic scales, the term “about” refers to the stated value plus or minus 0.3 log units, or plus or minus 0.2 log units, or plus or minus 0.1 log units. For example, a value of “about pH 4.6” can encompass a pH range of 4.5-4.7.

The term “substantially free of” or “substantially in the absence of” with respect to a composition refers to a composition that includes at least about 85 or 90% by weight, in certain embodiments at least about 95%, 98%, 99% or 100% by weight, of a designated enantiomer or stereoisomer of a compound. For example, “substantially free of” or “substantially in the absence of” with respect to a composition can refer to a composition that includes about 85%, 86%, 87%, 88%, 89%, 90%, 91%, 92%, 93%, 94%, 95%, 96%, 97%, 98%, 99%, or 100% by weight of a designated enantiomer or stereoisomer of a compound. In certain embodiments, in the methods and compounds provided herein, the compounds are substantially free of other enantiomers or stereoisomers.

Similarly, the term “isolated” with respect to a composition refers to a composition that includes at least 85, 90%, 95%, 98%, and 99% to 100% by weight, of a designated compound, enantiomer, or stereoisomer, the remainder comprising other chemical species, enantiomers, or stereoisomers. For example, “isolated” with respect to a composition can refer to a composition that includes about 85%, 86%, 87%, 88%, 89%, 90%, 91%, 92%, 93%, 94%, 95%, 96%, 97%, 98%, 99%, or 100% by weight of a designated compound, enantiomer, or stereoisomer, the remainder comprising other chemical species, enantiomers, or stereoisomers.

As used herein, the terms “subject” and “patient” are used interchangeably herein. The terms “subject” and “subjects” refer to an animal, such as a mammal including a non-primate (e.g., a cow, pig, horse, cat, dog, rat, and mouse) and a primate (e.g., a monkey such as a cynomolgous monkey, a chimpanzee and a human), and for example, a human. In another embodiment, the subject is a farm animal (e.g., a horse, a cow, a pig, etc.) or a pet (e.g., a dog or a cat). In certain embodiments, the subject is a human.

“Therapeutically effective amount” refers to an amount of a compound or composition that, when administered to a subject for treating a disease, is sufficient to effect such treatment for the disease. A “therapeutically effective amount” can vary depending on, inter alia, the compound, the disease and its severity, and the age, weight, etc., of the subject to be treated.

“Treating” or “treatment” of any disease or disorder refers, in certain embodiments, to ameliorating a disease or disorder that exists in a subject. In another embodiment, “treating” or “treatment” includes ameliorating at least one physical parameter, which may be indiscernible by the subject. In yet another embodiment, “treating” or “treatment” includes modulating the disease or disorder, either physically (e.g., stabilization of a discernible symptom) or physiologically (e.g., stabilization of a physical parameter) or both. In yet another embodiment, “treating” or “treatment” includes delaying the onset of the disease or disorder.

As used herein, the terms “prophylactic agent” and “prophylactic agents” as used refer to any agent(s) which can be used in the prevention of a disorder or one or more symptoms thereof. In certain embodiments, the term “prophylactic agent” includes a compound provided herein. In certain other embodiments, the term “prophylactic agent” does not refer a compound provided herein. For example, a prophylactic agent is an agent which is known to be useful for, or has been or is currently being used to prevent or impede the onset, development, progression and/or severity of a disorder.

As used herein, the phrase “prophylactically effective amount” refers to the amount of a therapy (e.g., prophylactic agent) which is sufficient to result in the prevention or reduction of the development, recurrence or onset of one or more symptoms associated with a disorder (, or to enhance or improve the prophylactic effect(s) of another therapy (e.g., another prophylactic agent).

### L-Ergothioneine Phosphoric Acid Co-Crystal Form A

In one embodiment, provided herein is an isolated crystalline form of L-Ergothioneine phosphoric acid. In one embodiment, the crystalline form is co-crystal L-Ergothioneine phosphoric acid Form A.

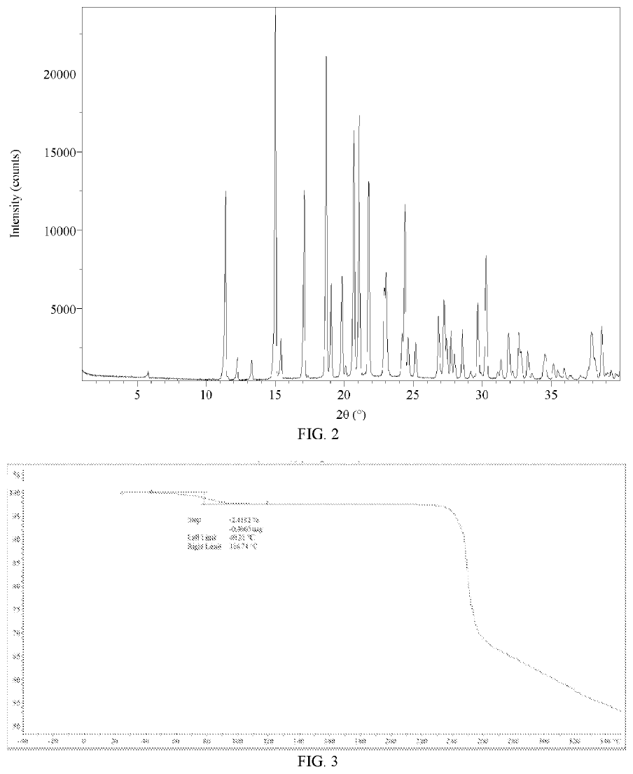

In one embodiment, co-crystal L-Ergothioneine phosphoric acid Form A is characterized by an X-ray powder diffraction (XRPD) pattern substantially similar to that set forth in FIG. 2. In one embodiment, co-crystal L-Ergothioneine phosphoric acid Form A is characterized by an X-ray powder diffraction (XRPD) pattern comprising peaks substantially similar to those set forth in Table 5. In certain embodiments, co-crystal L-Ergothioneine phosphoric acid Form A is characterized by an XRPD pattern comprising:
a) 2θ values of about 11.38, 14.99, 15.41, 18.68, 19.05, 19.82, 20.69, 21.07, 21.78, 24.38, 24.63, 25.17, 27.22, 27.73, 28.54, and 29.68±0.2° 2θ;
b) 2θ values including at least or selected from about 11.38, 14.99, 15.41, 18.68, 19.05, 19.82, 20.69, 21.07, 21.78, 24.38, 24.63, 25.17, 27.22, 27.73, 28.54, and 29.68±0.2° 2θ;
c) at least two, three, four, or five 2θ values selected from about 11.38, 14.99, 15.41, 18.68, 19.05, 19.82, 20.69, 21.07, 21.78, 24.38, 24.63, 25.17, 27.22, 27.73, 28.54, and 29.68±0.2° 2θ;
d) at least six, seven, or eight 2θ values selected from about 11.38, 14.99, 15.41, 18.68, 19.05, 19.82, 20.69, 21.07, 21.78, 24.38, 24.63, 25.17, 27.22, 27.73, 28.54, and 29.68±0.2° 2θ;
e) 2θ values of about 11.38, 14.99, 18.68, 20.69, 21.07, and 21.78±0.2° 2θ;
f) 2θ values including at least or selected from about 11.38, 14.99, 18.68, 20.69, 21.07, and 21.78±0.2° 2θ;
g) 2θ values of about 14.99, 18.68, 20.69, and 21.07±0.2° 2θ;
h) 2θ values including at least or selected from about 14.99, 18.68, 20.69, and 21.07±0.2° 2θ;
i) 2θ values of about 14.99, 18.68, and 21.07±0.2° 2θ;
j) 2θ values including at least or selected from about 14.99, 18.68, and 21.07±0.2° 2θ;
k) 2θ values of about 11.38, 14.99, 15.41, 19.82, 20.69, 21.78, 24.63, 25.17, 27.22, 27.73, 28.54, and 29.68±0.2° 2θ;
l) 2θ values including at least or selected from about 11.38, 14.99, 15.41, 19.82, 20.69, 21.78, 24.63, 25.17, 27.22, 27.73, 28.54, and 29.68±0.2° 2θ;
m) 2θ values of about 11.38, 14.99, 19.82, 20.69, 21.78, and 27.22±0.2° 2θ;
n) 2θ values including at least or selected from about 11.38, 14.99, 19.82, 20.69, 21.78, and 27.22±0.2° 2θ;
o) 2θ values of about 11.38, 14.99, 20.69, and 21.78±0.2° 2θ;
p) 2θ values including at least or selected from about 11.38, 14.99, 20.69, and 21.78±0.2° 2θ;
q) 2θ values of about 11.38, 14.99, 18.68, 19.82, 21.78, 24.38, and 27.22±0.2° 2θ;
r) 2θ values including at least or selected from about 11.38, 14.99, 18.68, 19.82, 21.78, 24.38, and 27.22±0.2° 2θ;
s) 2θ values of about 11.38, 14.99, 18.68, and 21.78±0.2° 2θ;
t) 2θ values including at least or selected from about 11.38, 14.99, 18.68, and 21.78±0.2° 2θ;
u) 2θ values of about 14.99, 20.69, 21.07, and 25.17±0.2° 2θ; and
v) 2θ values including at least or selected from about 14.99, 20.69, 21.07, and 25.17±0.2° 2θ.

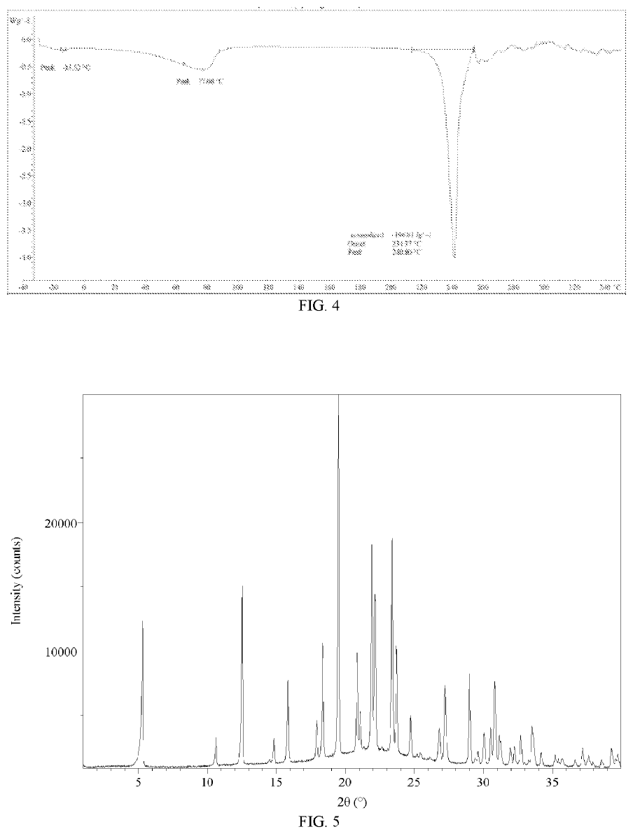

In one embodiment, co-crystal L-Ergothioneine phosphoric acid Form A is characterized by a differential scanning calorimetry (DSC) thermograph having two endotherms wherein the first has an endotherm maximum between about 75° C. and 85° C. and wherein the second has an endotherm maximum between about 235° C. and 245° C. In one embodiment, co-crystal L-Ergothioneine phosphoric acid Form A is characterized by a differential scanning calorimetry (DSC) thermograph having two endotherms wherein the first has an endotherm maximum at about 77° C. and wherein the second has an endotherm maximum at about 241° C. In one embodiment, co-crystal L-Ergothioneine phosphoric acid Form A is characterized by a DSC thermograph substantially similar to that set forth in FIG. 4.

In one embodiment, co-crystal L-Ergothioneine phosphoric acid Form A is characterized by a weight loss in the range of about 1% and 5% when heated up to about 120° C. in a thermogravimetric analysis (TGA). In one embodiment, co-crystal L-Ergothioneine phosphoric acid Form A is characterized by a weight loss of about 2.4% when heated up to about 117° C. in a TGA. In one embodiment, co-crystal L-Ergothioneine phosphoric acid Form A is characterized by a TGA substantially similar to that set forth in FIG. 3.

Co-crystal L-Ergothioneine phosphoric acid Form A can be synthesized as described in Example 1. In certain embodiments, Form A is synthesized by mixing L-Ergothioneine in a protic solvent. In certain embodiments, the solvent is a polar protic solvent. In certain embodiments, the solvent is selected from the group consisting of methanol, ethanol, isopropanol, formic acid, and acetic acid. In certain embodiments, the mixture comprises water. In certain embodiments, the mixture does not comprise water. In certain embodiments, a sufficient amount of phosphoric acid is added to afford the precipitation of L-Ergothioneine phosphoric acid. In certain embodiments, the amount is at least 1.0 molar equivalent. In certain embodiments, the amount is about 1.0 molar equivalent. After standing, for instance overnight, the precipitate is filtered and dried to afford co-crystal L-Ergothioneine phosphoric acid Form A as an opaque, white solid.

In one embodiment, co-crystal L-Ergothioneine phosphoric acid Form A is synthesized by a method comprising:
a) mixing L-Ergothioneine in acetic acid;
b) adding at least 1.0 molar equivalent of phosphoric acid to afford a precipitate;
c) isolating the precipitate to afford co-crystal L-Ergothioneine phosphoric acid Form A.

In one embodiment, the precipitate is allowed to stand for at least 12 hours, at least 14 hours, at least 16 hours, at least 18 hours, at least 20 hours, or at least 24 hours in step (c).

### L-Ergothioneine Acetate Form A

In one embodiment, provided herein is an isolated crystalline form of L-Ergothioneine acetate Form A.

In one embodiment, L-Ergothioneine acetate Form A is characterized by an X-ray powder diffraction (XRPD) pattern substantially similar to that set forth in FIG. 5. In one embodiment, L-Ergothioneine acetate Form A is characterized by an X-ray powder diffraction (XRPD) pattern comprising peaks substantially similar to those set forth in Table 6. In certain embodiments, L-Ergothioneine acetate Form A is characterized by an XRPD pattern comprising:
a) 2θ values of about 5.30, 10.63, 12.52, 14.82, 15.83, 17.93, 18.35, 19.50, 20.84, 21.06, 21.91, 22.16, 23.40, 23.71, 24.73, 29.00, 30.05, and 30.53±0.2° 2θ;
b) 2θ values including at least or selected from about 5.30, 10.63, 12.52, 14.82, 15.83, 17.93, 18.35, 19.50, 20.84, 21.06, 21.91, 22.16, 23.40, 23.71, 24.73, 29.00, 30.05, and 30.53±0.2° 2θ;
c) at least two, three, four, or five 2θ values selected from about 5.30, 10.63, 12.52, 14.82, 15.83, 17.93, 18.35, 19.50, 20.84, 21.06, 21.91, 22.16, 23.40, 23.71, 24.73, 29.00, 30.05, and 30.53±0.2° 2θ;
d) at least six, seven, or eight 2θ values selected from about 5.30, 10.63, 12.52, 14.82, 15.83, 17.93, 18.35, 19.50, 20.84, 21.06, 21.91, 22.16, 23.40, 23.71, 24.73, 29.00, 30.05, and 30.53±0.2° 2θ;
e) 2θ values of about 5.30, 12.52, 19.50, 20.84, 21.91, 22.16, 23.40, and 23.71±0.2° 2θ; and
f) 2θ values including at least or selected from 5.30, 12.52, 19.50, 20.84, 21.91, 22.16, 23.40, and 23.71±0.2° 2θ.

In one embodiment, L-Ergothioneine acetate Form A is characterized by a differential scanning calorimetry (DSC) thermograph having three endotherms with endotherm maxima between about 100° C. and 110° C., between about 180° C. and 190° C., and between about 260° C. and 270° C. In one embodiment, L-Ergothioneine acetate Form A is characterized by a differential scanning calorimetry (DSC) thermograph having three endotherms with endotherm maxima of about 106° C., 182° C., and 265° C.

In one embodiment, L-Ergothioneine acetate Form A is characterized by a differential scanning calorimetry (DSC) thermograph having three endotherms wherein the first has an endotherm maximum between about 100° C. and 110° C., the second endotherm exhibits an onset between about 175° C. and 185° C., and third endotherm exhibits an onset between about 255° C. and 265° C. In one embodiment, L-Ergothioneine acetate Form A is characterized by a differential scanning calorimetry (DSC) thermograph having three endotherms wherein the first has an endotherm maximum at about 106° C., the second endotherm exhibits an onset of about 180° C., and third endotherm exhibits an onset of about 258° C. In one embodiment, L-Ergothioneine acetate Form A is characterized by DSC thermograph substantially similar to that set forth in FIG. 7.

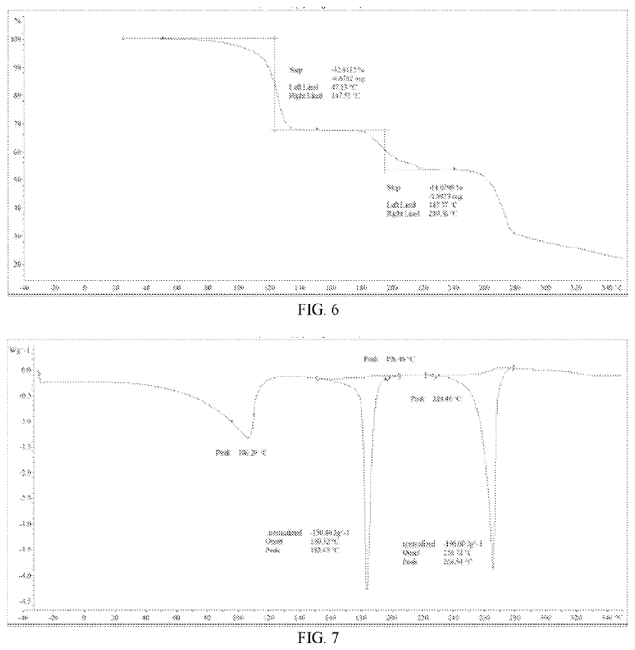

In one embodiment, L-Ergothioneine acetate Form A is characterized by a weight loss in the range of about 30% to 40% when heated to about 150° C. and an additional weight loss in the range of about 10% to 20% when heated from about 150° C. to 240° C. in a thermogravimetric analysis (TGA). In one embodiment, L-Ergothioneine acetate Form A is characterized by a weight loss of about 33% when heated to about 148° C. and an additional weight loss of about 14% when further heated to about 239° C. in a TGA. In one embodiment, L-Ergothioneine acetate Form A is characterized by a TGA substantially similar to that set forth in FIG. 6.

### L-Ergothioneine Benzoate Form A

In one embodiment, provided herein is an isolated crystalline form of L-Ergothioneine benzoate Form A.

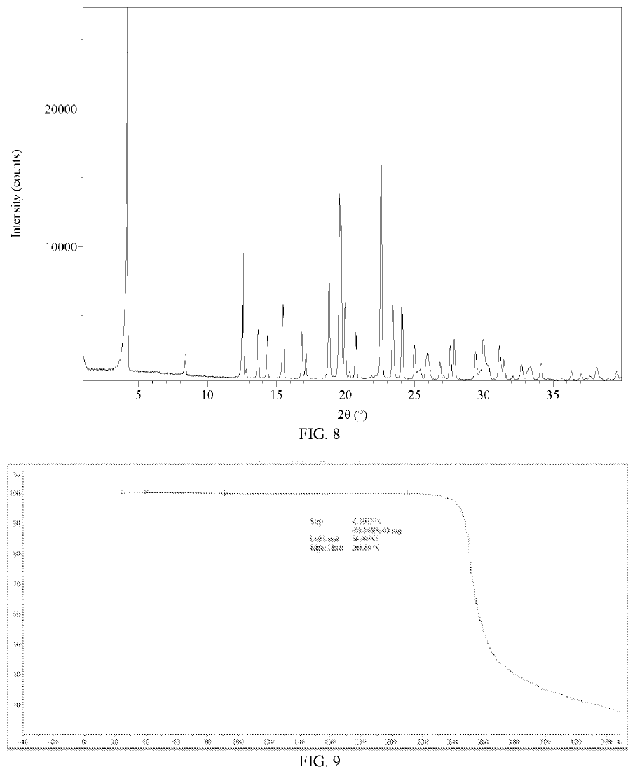

In one embodiment, L-Ergothioneine benzoate Form A is characterized by an X-ray powder diffraction (XRPD) pattern substantially similar to that set forth in FIG. 8. In one embodiment, L-Ergothioneine benzoate Form A is characterized by an X-ray powder diffraction (XRPD) pattern comprising peaks substantially similar to those set forth in Table 7. In certain embodiments, L-Ergothioneine benzoate Form A is characterized by an XRPD pattern comprising:
a) 2θ values of about 4.20, 8.41, 12.55, 13.67, 14.34, 15.48, 17.13, 18.80, 19.95, 20.74, 22.56, 23.41, 24.08, 27.58, and 27.88±0.2° 2θ;
b) 2θ values including at least or selected from about 4.20, 8.41, 12.55, 13.67, 14.34, 15.48, 17.13, 18.80, 19.95, 20.74, 22.56, 23.41, 24.08, 27.58, and 27.88±0.2° 2θ;
c) at least two, three, four, or five 2θ values selected from about 4.20, 8.41, 12.55, 13.67, 14.34, 15.48, 17.13, 18.80, 19.95, 20.74, 22.56, 23.41, 24.08, 27.58, and 27.88±0.2° 2θ;
d) at least six, seven, or eight 2θ values selected from about 4.20, 8.41, 12.55, 13.67, 14.34, 15.48, 17.13, 18.80, 19.95, 20.74, 22.56, 23.41, 24.08, 27.58, and 27.88±0.2° 2θ;
e) 2θ values of about 4.20, 12.55, 18.80, 19.95, 22.56, and 24.08±0.2° 2θ; and
f) 2θ values including at least or selected from about 4.20, 12.55, 18.80, 19.95, 22.56, and 24.08±0.2° 2θ.

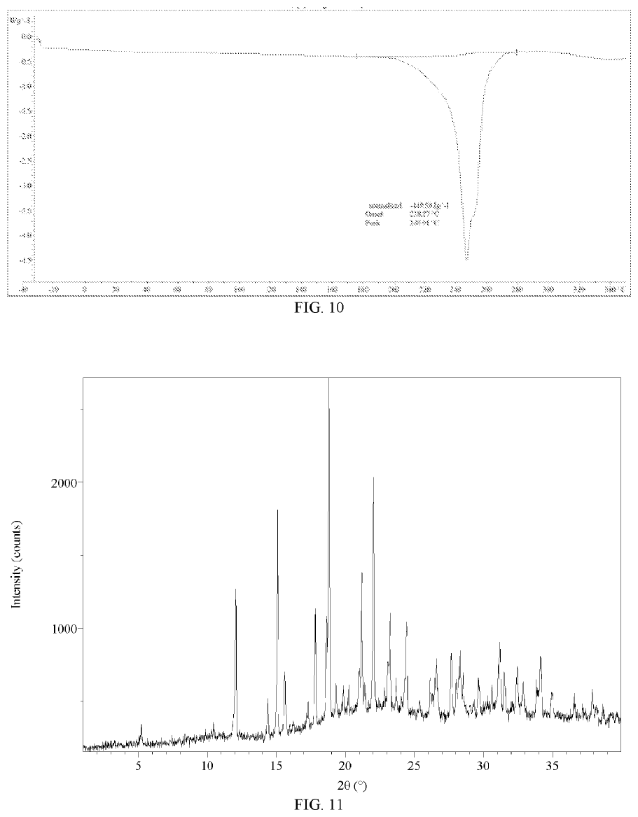

In one embodiment, L-Ergothioneine benzoate Form A is characterized by a differential scanning calorimetry (DSC) thermograph having one endotherm with an onset between about 230° C. and 250° C. In one embodiment, L-Ergothioneine benzoate Form A is characterized by a differential scanning calorimetry (DSC) thermograph having one endotherm with an onset at about 238° C. In one embodiment, L-Ergothioneine benzoate Form A is characterized by DSC thermograph substantially similar to that set forth in FIG. 10.

In one embodiment, L-Ergothioneine benzoate Form A is characterized by a weight loss in the range of 0% to about 1% when heated to about 215° C. in a thermogravimetric analysis (TGA). In one embodiment, L-Ergothioneine benzoate Form A is characterized by a weight loss of about 0.4% when heated to about 209° C. in a TGA. In one embodiment, L-Ergothioneine benzoate Form A is characterized by a TGA substantially similar to that set forth in FIG. 9.

### L-Ergothioneine Calcium Chloride Material A

In one embodiment, provided herein is an isolated form of L-Ergothioneine calcium chloride material A.

In one embodiment, L-Ergothioneine calcium chloride Form A is characterized by an X-ray powder diffraction (XRPD) pattern substantially similar to that set forth in FIG. 11. In one embodiment, L-Ergothioneine calcium chloride material A is characterized by an X-ray powder diffraction (XRPD) pattern comprising peaks substantially similar to those set forth in Table 8. In certain embodiments, L-Ergothioneine calcium chloride material A is characterized by an XRPD pattern comprising:
a) 2θ values of about 12.07, 15.09, 17.83, 18.80, 21.19, 22.04, 23.23, and 24.43±0.2° 2θ;
b) 2θ values including at least or selected from 12.07, 15.09, 17.83, 18.80, 21.19, 22.04, 23.23, and 24.43 about ±0.2° 2θ;
c) at least two, three, four, or five 2θ values selected from about 12.07, 15.09, 17.83, 18.80, 21.19, 22.04, 23.23, and 24.43±0.2° 2θ;
d) at least six, seven, or eight 2θ values selected from about 12.07, 15.09, 17.83, 18.80, 21.19, 22.04, 23.23, and 24.43±0.2° 2θ;
e) 2θ values of about ±about 15.09, 18.80, 21.19, and 22.04 0.2° 2θ; and
f) 2θ values including at least or selected from about 15.09, 18.80, 21.19, and 22.04±0.2° 2θ.

### L-Ergothioneine Hemifumarate Form A

In one embodiment, provided herein is an isolated crystalline form of L-Ergothioneine hemifumarate Form A.

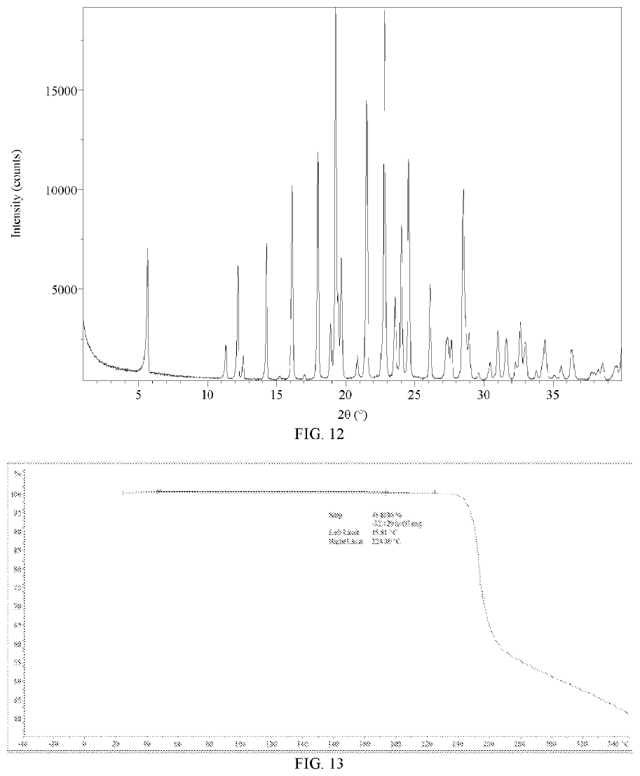

In one embodiment, L-Ergothioneine hemifumarate Form A is characterized by an X-ray powder diffraction (XRPD) pattern substantially similar to that set forth in FIG. 12. In one embodiment, L-Ergothioneine hemifumarate Form A is characterized by an X-ray powder diffraction (XRPD) pattern comprising peaks substantially similar to those set forth in Table 9. In certain embodiments, L-Ergothioneine hemifumarate Form A is characterized by an XRPD pattern comprising:
a) 2θ values of about 5.65, 11.32, 12.18, 14.27, 16.13, 17.98, 18.90, 19.27, 19.69, 21.51, 22.83, 23.56, 24.03, 24.57, and 26.12±0.2° 2θ;
b) 2θ values including at least or selected from about 5.65, 11.32, 12.18, 14.27, 16.13, 17.98, 18.90, 19.27, 19.69, 21.51, 22.83, 23.56, 24.03, 24.57, and 26.12±0.2° 2θ;
c) at least two, three, four, or five 2θ values selected from about 5.65, 11.32, 12.18, 14.27, 16.13, 17.98, 18.90, 19.27, 19.69, 21.51, 22.83, 23.56, 24.03, 24.57, and 26.12±0.2° 2θ;
d) at least six, seven, or eight 2θ values selected from about 5.65, 11.32, 12.18, 14.27, 16.13, 17.98, 18.90, 19.27, 19.69, 21.51, 22.83, 23.56, 24.03, 24.57, and 26.12±0.2° 2θ;
e) 2θ values of about 16.13, 17.98, 19.27, 21.51, 22.83, and 24.57±0.2° 2θ; and
f) 2θ values including at least or selected from about 16.13, 17.98, 19.27, 21.51, 22.83, and 24.57±0.2° 2θ.

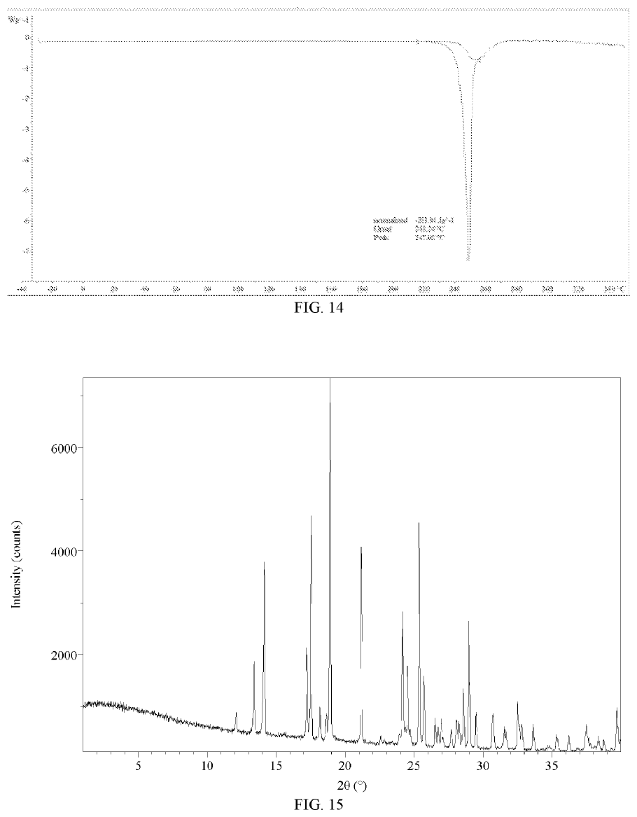

In one embodiment, L-Ergothioneine hemifumarate Form A is characterized by a differential scanning calorimetry (DSC) thermograph having one endotherm with an onset between about 240° C. and 250° C. In one embodiment, L-Ergothioneine hemifumarate Form A is characterized by a differential scanning calorimetry (DSC) thermograph having one endotherm with an onset at about 244° C. In one embodiment, L-Ergothioneine hemifumarate Form A is characterized by DSC thermograph substantially similar to that set forth in FIG. 14.

In one embodiment, L-Ergothioneine hemifumarate Form A is characterized by a weight loss in the range of 0% to about 1% when heated to about 230° C. in a thermogravimetric analysis (TGA). In one embodiment, L-Ergothioneine hemifumarate Form A is characterized by a weight loss of about 0.4% when heated to about 224° C. in a TGA. In one embodiment, L-Ergothioneine hemifumarate Form A is characterized by a TGA substantially similar to that set forth in FIG. 13.

### L-Ergothioneine Hydrochloride Form A

In one embodiment, provided herein is an isolated crystalline form of L-Ergothioneine hydrochloride Form A.

In one embodiment, L-Ergothioneine hydrochloride Form A is characterized by an X-ray powder diffraction (XRPD) pattern substantially similar to that set forth in FIG. 15. In one embodiment, L-Ergothioneine hydrochloride Form A is characterized by an X-ray powder diffraction (XRPD) pattern comprising peaks substantially similar to those set forth in Table 11. In certain embodiments, L-Ergothioneine hydrochloride Form A is characterized by an XRPD pattern comprising:
a) 2θ values of about 12.10, 13.39, 14.13, 17.22, 17.52, 18.17, 18.91, 21.16, 24.17, 24.52, 25.37, and 28.97±0.2° 2θ;
b) 2θ values including at least or selected from about 12.10, 13.39, 14.13, 17.22, 17.52, 18.17, 18.91, 21.16, 24.17, 24.52, 25.37, and 28.97±0.2° 2θ;
c) at least two, three, four, or five 2θ values selected from about 12.10, 13.39, 14.13, 17.22, 17.52, 18.17, 18.91, 21.16, 24.17, 24.52, 25.37, and 28.97±0.2° 2θ;
d) at least six, seven, or eight 2θ values selected from about 12.10, 13.39, 14.13, 17.22, 17.52, 18.17, 18.91, 21.16, 24.17, 24.52, 25.37, and 28.97±0.2° 2θ;
e) 2θ values of about 14.13, 17.52, 18.91, 21.16, 24.17, and 25.37±0.2° 2θ; and
f) 2θ values including at least or selected from about 14.13, 17.52, 18.91, 21.16, 24.17, and 25.37±0.2° 2θ.

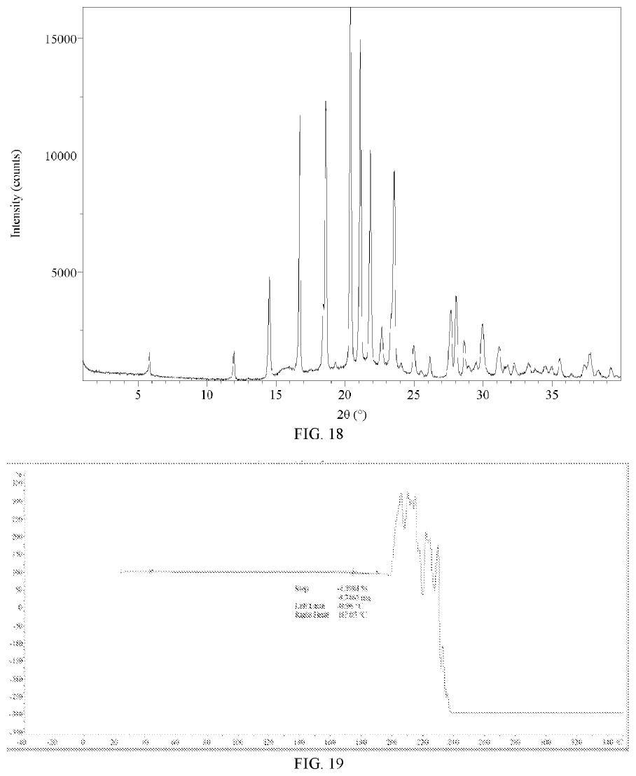

In one embodiment, L-Ergothioneine hydrochloride Form A is characterized by a differential scanning calorimetry (DSC) thermograph having two endotherms with endotherm maxima between about 125° C. and 135° C. and between about 195° C. and 205° C. In one embodiment, L-Ergothioneine hydrochloride Form A is characterized by a differential scanning calorimetry (DSC) thermograph having two endotherms with endotherm maxima at about 131° C. and 201° C. In one embodiment, L-Ergothioneine hydrochloride Form A is characterized by DSC thermograph substantially similar to that set forth in FIG. 17.

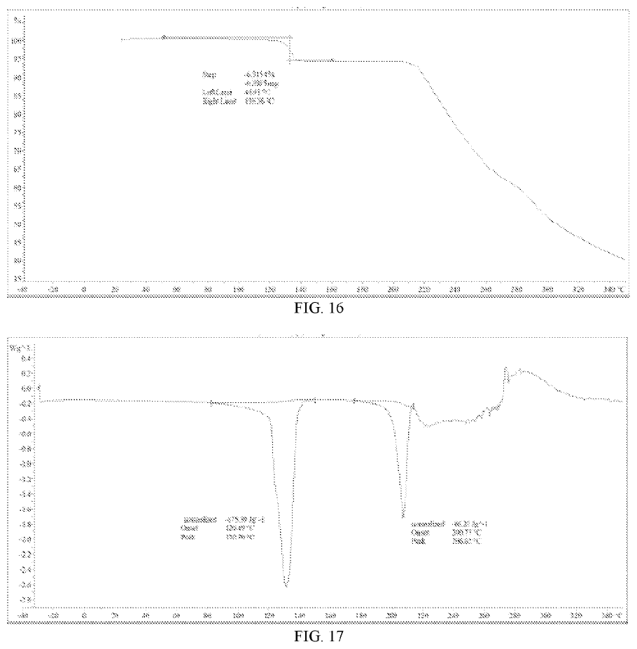

In one embodiment, L-Ergothioneine hydrochloride Form A is characterized by a weight loss in the range of about 3% and 8% when heated up to about 160° C. in a thermogravimetric analysis (TGA). In one embodiment, L-Ergothioneine hydrochloride Form A is characterized by a weight loss of about 6.3% when heated up to about 158° C. in a TGA. In one embodiment, L-Ergothioneine hydrochloride Form A is characterized by a TGA substantially similar to that set forth in FIG. 16.

### L-Ergothioneine Sulfate Form A

In one embodiment, provided herein is an isolated crystalline form of L-Ergothioneine sulfate Form A.

In one embodiment, L-Ergothioneine sulfate Form A is characterized by an X-ray powder diffraction (XRPD) pattern substantially similar to that set forth in FIG. 18. In one embodiment, L-Ergothioneine sulfate Form A is characterized by an X-ray powder diffraction (XRPD) pattern comprising peaks substantially similar to those set forth in Table 12. In certain embodiments, L-Ergothioneine sulfate Form A is characterized by an XRPD pattern comprising:
a) 2θ values of about 5.83, 11.97, 14.52, 16.71, 18.60, 20.41, 21.09, 21.83, 22.65, and 28.04±0.2° 2θ;
b) 2θ values including at least or selected from about 5.83, 11.97, 14.52, 16.71, 18.60, 20.41, 21.09, 21.83, 22.65, and 28.04±0.2° 2θ;
c) at least two, three, four, or five 2θ values selected from about 5.83, 11.97, 14.52, 16.71, 18.60, 20.41, 21.09, 21.83, 22.65, and 28.04±0.2° 2θ;
d) at least six, seven, or eight 2θ values selected from about 5.83, 11.97, 14.52, 16.71, 18.60, 20.41, 21.09, 21.83, 22.65, and 28.04±0.2° 2θ;
e) 2θ values of about 16.71, 18.60, 20.41, and 21.09±0.2° 2θ; and
f) 2θ values including at least or selected from about 16.71, 18.60, 20.41, and 21.09±0.2° 2θ.

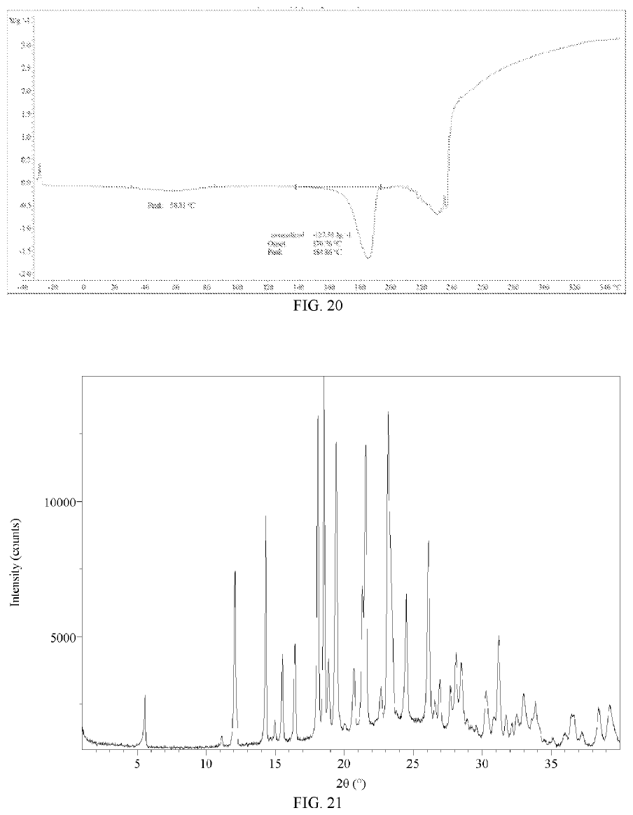

In one embodiment, L-Ergothioneine sulfate Form A is characterized by a differential scanning calorimetry (DSC) thermograph having one endotherm with an onset between about 165° C. and 175° C. In one embodiment, L-Ergothioneine sulfate Form A is characterized by a differential scanning calorimetry (DSC) thermograph having one endotherm with an onset at about 171° C. In one embodiment, L-Ergothioneine sulfate Form A is characterized by DSC thermograph substantially similar to that set forth in FIG. 20.

In one embodiment, L-Ergothioneine sulfate Form A is characterized by a weight loss in the range of about 2% and 8% when heated up to about 190° C. in a thermogravimetric analysis (TGA). In one embodiment, L-Ergothioneine sulfate Form A is characterized by a weight loss of about 4.3% when heated up to about 187° C. in a TGA. In one embodiment, L-Ergothioneine sulfate Form A is characterized by a TGA substantially similar to that set forth in FIG. 19.

### L-Ergothioneine Hemitartrate Form a

In one embodiment, provided herein is an isolated crystalline form of L-Ergothioneine hemitartrate Form A.

In one embodiment, L-Ergothioneine hemitartrate Form A is characterized by an X-ray powder diffraction (XRPD) pattern substantially similar to that set forth in FIG. 21. In one embodiment, L-Ergothioneine hemitartrate Form A is characterized by an X-ray powder diffraction (XRPD) pattern comprising peaks substantially similar to those set forth in Table 13. In certain embodiments, L-Ergothioneine hemitartrate Form A is characterized by an XRPD pattern comprising:
a) 2θ values of about 5.55, 14.32, 15.53, 16.41, 18.10, 18.53, 18.85, 20.69, 26.56, and 26.92±0.2° 2θ;
b) 2θ values including at least or selected from about 5.55, 14.32, 15.53, 16.41, 18.10, 18.53, 18.85, 20.69, 26.56, and 26.92±0.2° 2θ;
c) at least two, three, four, or five 2θ values selected from about 5.55, 14.32, 15.53, 16.41, 18.10, 18.53, 18.85, 20.69, 26.56, and 26.92±0.2° 2θ;
d) at least six, seven, or eight 2θ values selected from about 5.55, 14.32, 15.53, 16.41, 18.10, 18.53, 18.85, 20.69, 26.56, and 26.92±0.2° 2θ;
e) 2θ values of about 14.32, 15.53, 16.41, 18.10, and 18.53±0.2° 2θ; and
f) 2θ values including at least or selected from about 14.32, 15.53, 16.41, 18.10, and 18.53±0.2° 2θ.

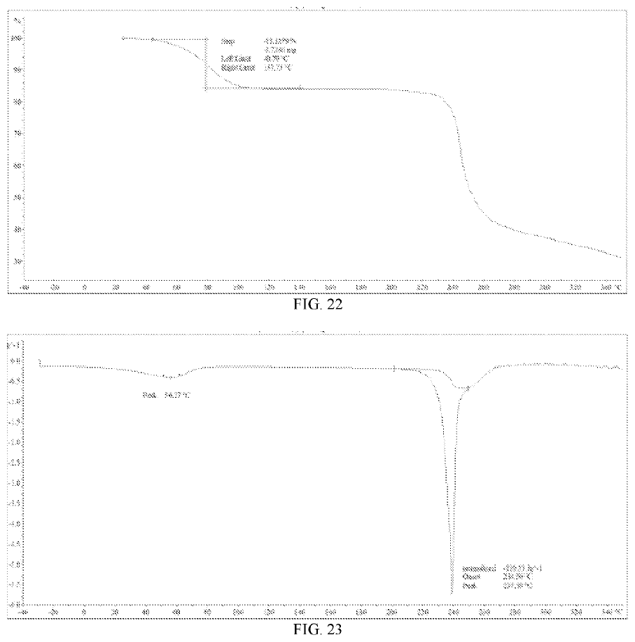

In one embodiment, L-Ergothioneine hemitartrate Form A is characterized by a differential scanning calorimetry (DSC) thermograph comprising two endotherms with endotherm maxima between about 50° C. and 60° C. and between about 235° C. and 245° C. In one embodiment, L-Ergothioneine hemitartrate Form A is characterized by a differential scanning calorimetry (DSC) thermograph comprising two endotherms with endotherm maxima of about 56° C. and about 234° C. In one embodiment, L-Ergothioneine hemitartrate Form A is characterized by DSC thermograph substantially similar to that set forth in FIG. 23.

In one embodiment, L-Ergothioneine hemitartrate Form A is characterized by a weight loss in the range of about 10% and 20% when heated up to about 140° C. in a thermogravimetric analysis (TGA). In one embodiment, L-Ergothioneine hemitartrate Form A is characterized by a weight loss of about 15% when heated up to about 137° C. in a TGA. In one embodiment, L-Ergothioneine hemifumarate Form A is characterized by a TGA substantially similar to that set forth in FIG. 22.

L-Ergothioneine Form C Anhydrate

In one embodiment, provided herein is an isolated crystalline form of the anhydrate free base L-Ergothioneine (Form C).

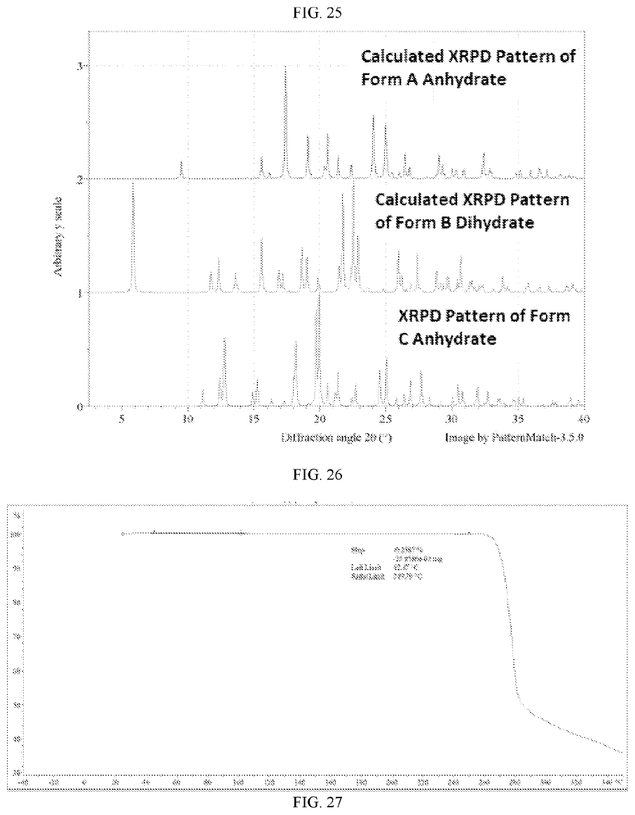

In one embodiment, L-Ergothioneine Form C anhydrate is characterized by an X-ray powder diffraction (XRPD) pattern substantially similar to that set forth in FIG. 25. In one embodiment, L-Ergothioneine Form C anhydrate is characterized by an X-ray powder diffraction (XRPD) pattern comprising peaks substantially similar to those set forth in Table 15. In certain embodiments, L-Ergothioneine Form C anhydrate is characterized by an XRPD pattern comprising:
a) 2θ values of about 11.17, 12.44, 12.77, 14.90, 15.26, 19.72, 19.96, 20.59, 21.16, 21.36, 22.72, 25.04, 26.88, 27.70, and 30.47±0.2° 2θ;
b) 2θ values including at least or selected from about 11.17, 12.44, 12.77, 14.90, 15.26, 19.72, 19.96, 20.59, 21.16, 21.36, 22.72, 25.04, 26.88, 27.70, and 30.47±0.2° 2θ;
c) at least two, three, four, or five 2θ values selected from about 11.17, 12.44, 12.77, 14.90, 15.26, 19.72, 19.96, 20.59, 21.16, 21.36, 22.72, 25.04, 26.88, 27.70, and 30.47±0.2° 2θ;
d) at least six, seven, or eight 2θ values selected from about 11.17, 12.44, 12.77, 14.90, 15.26, 19.72, 19.96, 20.59, 21.16, 21.36, 22.72, 25.04, 26.88, 27.70, and 30.47±0.2° 2θ;
e) 2θ values of about 12.44, 12.77, 19.72, 19.96, 21.36, and 25.04±0.2° 2θ;
f) 2θ values including at least or selected from 12.44, 12.77, 19.72, 19.96, 21.36, and 25.04±0.2° 2θ;
g) 2θ values of about 11.17, 12.44, 12.77, 19.72, 19.96, and 22.72±0.2° 2θ;
h) 2θ values including at least or selected from about 11.17, 12.44, 12.77, 19.72, 19.96, and 22.72±0.2° 2θ; and
i) 2θ values of about 11.17 and 12.77±0.2° 2θ;

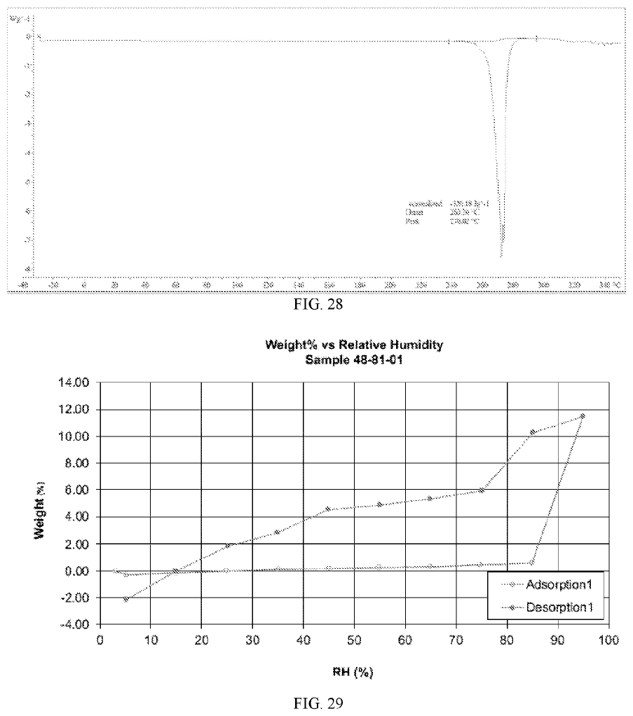

In one embodiment, L-Ergothioneine Form C anhydrate is characterized by a differential scanning calorimetry (DSC) thermograph having an endotherm with an onset between about 260° C. and 270° C. In one embodiment, L-Ergothioneine Form C anhydrate is characterized by a DSC thermograph having an endotherm with an onset of about 265° C. In one embodiment, L-Ergothioneine Form C anhydrate is characterized by DSC thermograph substantially similar to that set forth in FIG. 28.

In one embodiment, L-Ergothioneine Form C anhydrate is characterized by a weight loss in the range of 0% to about 1% when heated up to about 255° C. in a thermogravimetric analysis (TGA). In one embodiment, L-Ergothioneine Form C anhydrate is characterized by a weight loss of about 0.3% when heated up to about 250° C. in a TGA. In one embodiment, L-Ergothioneine Form C anhydrate is characterized by a TGA substantially similar to that set forth in FIG. 27.

“Substantially” when describing XRPD patterns is meant that the reported peaks can vary by ±0.2°.

“Substantially” when describing differential scanning calorimetry (DSC) thermographs and a thermogravimetric analysis (TGA) is meant that the reported temperatures can vary by +0.5° C.

Also provided herein is a salt form of L-Ergothioneine selected from the group consisting of L-Ergothioneine acetate, L-Ergothioneine benzoate, L-Ergothioneine hemifumurate, L-Ergothioneine phosphoric acid, L-Ergothioneine sulfate, L-Ergothioneine hemitartrate, L-Ergothioneine hemimaleate, L-Ergothioneine maleate, L-Ergothioneine adipic acid, L-Ergothioneine L-aspartic acid, L-Ergothioneine citric acid, L-Ergothioneine camphorsulfonic acid, L-Ergothioneine methanesulfonic sulfonic acid, and L-Ergothioneine tosylate.

In one embodiment, provided herein is a salt form of L-Ergothioneine selected from the group consisting of L-Ergothioneine acetate, L-Ergothioneine benzoate, L-Ergothioneine hemifumurate, L-Ergothioneine phosphoric acid, L-Ergothioneine sulfate, and L-Ergothioneine hemitartrate.

In one embodiment, provided herein is a salt form of L-Ergothioneine selected from the group consisting of L-Ergothioneine hemifumurate, L-Ergothioneine hemitartrate, L-Ergothioneine hemimaleate, L-Ergothioneine adipic acid, L-Ergothioneine L-aspartic acid, and L-Ergothioneine citric acid.

The salt forms of L-Ergothioneine described herein can be in a form that is at least 90% free of the opposite D-Ergothioneine enantiomer (excluding the weight of the salt). In one embodiment, the salt forms of L-Ergothioneine is at least 95%, 98%, 99%, or even 100% free of the opposite D-Ergothioneine enantiomer (excluding the weight of the salt). For example, in certain embodiments, the L-Ergothioneine phosphoric acid is at least 90%, 95%, 98%, 99%, or even 100% free of the opposite D-Ergothioneine (excluding the weight of the salt).

Unless described otherwise the salt forms are substantially L-Ergothioneine. In an alternative embodiment, the salt form is a racemic mixture of L-Ergothioneine and D-Ergothioneine. In a further alternative embodiment, the salt form is in a form that is substantially the D-Ergothioneine. In one embodiment, the D-Ergothioneine is at least 90%, 95%, 98%, 99%, or even 100% free of the opposite L-Ergothioneine.

### Pharmaceutical Compositions

The forms of L-Ergothioneine provided herein, including the salt forms and crystalline forms thereof and the crystalline L-Ergothioneine Form C, can be formulated as pharmaceutical compositions using methods available in the art and those disclosed herein. Any of the compositions disclosed herein can be provided in the appropriate pharmaceutical composition and be administered by a suitable route of administration. The compositions provided herein can also be formulated as nutraceutical or nutritional formulations with additives such as nutraceutically or nutritionally acceptable excipients, nutraceutically or nutritionally acceptable carriers, and nutraceutically or nutritionally acceptable vehicles.

The methods provided herein encompass administering pharmaceutical or nutraceutical compositions containing at least one form of L-Ergothioneine provided herein, including the salt forms and crystalline forms thereof and the crystalline L-Ergothioneine Form C, either alone or in the form of a combination with one or more compatible and pharmaceutically acceptable carriers, such as diluents or adjuvants.

The term “nutraceutical” has been used to refer to any substance that is a food or a part of a food and provides medical or health benefits, including the prevention and treatment of disease. Hence, compositions falling under the label “nutraceutical” may range from isolated nutrients, dietary supplements and specific diets to genetically engineered designer foods, herbal products, and processed foods such as cereals, soups and beverages. In a more technical sense, the term has been used to refer to a product isolated or purified from foods, and generally sold in medicinal forms not usually associated with food and demonstrated to have a physiological benefit or provide protection against chronic disease.

In pharmaceutical compositions, use may be made, as solid compositions for oral administration, of tablets, pills, hard gelatin capsules, powders or granules. In these compositions, the active product is mixed with one or more inert diluents or adjuvants, such as sucrose, lactose or starch.

In clinical practice the compositions provided herein may be administered by any conventional route, in particular orally, parenterally, rectally or by inhalation (e.g. in the form of aerosols). In certain embodiments, the compositions provided herein are administered orally.

These compositions can comprise substances other than diluents, for example a lubricant, such as magnesium stearate, or a coating intended for controlled release.

In certain embodiments, a composition provided herein is a pharmaceutical composition or a single unit dosage form. Pharmaceutical compositions and single unit dosage forms provided herein comprise a prophylactically or therapeutically effective amount of one or more prophylactic or therapeutic agents (e.g., a compound provided herein, or other prophylactic or therapeutic agent), and a typically one or more pharmaceutically acceptable carriers or excipients. In a specific embodiment and in this context, the term “pharmaceutically acceptable” means approved by a regulatory agency of the Federal or a state government or listed in the U.S. Pharmacopeia or other generally recognized pharmacopeia for use in animals, and more particularly in humans. The term “carrier” includes a diluent, adjuvant (e.g., Freund's adjuvant (complete and incomplete)), excipient, or vehicle with which the therapeutic is administered. Such pharmaceutical carriers can be sterile liquids, such as water and oils, including those of petroleum, animal, vegetable or synthetic origin, such as peanut oil, soybean oil, mineral oil, sesame oil and the like. Water can be used as a carrier when the pharmaceutical composition is administered intravenously. Saline solutions and aqueous dextrose and glycerol solutions can also be employed as liquid carriers, particularly for injectable solutions. Examples of suitable pharmaceutical carriers are described in “Remington's Pharmaceutical Sciences” by E. W. Martin.

Typical pharmaceutical compositions and dosage forms comprise one or more excipients. Suitable excipients are well-known to those skilled in the art of pharmacy, and non-limiting examples of suitable excipients include starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol and the like. Whether a particular excipient is suitable for incorporation into a pharmaceutical composition or dosage form depends on a variety of factors well known in the art including, but not limited to, the way in which the dosage form will be administered to a subject and the specific active ingredients in the dosage form. The composition or single unit dosage form, if desired, can also contain minor amounts of wetting or emulsifying agents, or pH buffering agents.

Lactose free compositions provided herein can comprise excipients that are well known in the art and are listed, for example, in the U.S. Pharmacopeia (USP) SP (XXI)/NF (XVI). In general, lactose free compositions comprise an active ingredient, a binder/filler, and a lubricant in pharmaceutically compatible and pharmaceutically acceptable amounts. Exemplary lactose free dosage forms comprise an active ingredient, microcrystalline cellulose, pre gelatinized starch, and magnesium stearate.

Further encompassed herein are anhydrous pharmaceutical compositions and dosage forms comprising active ingredients, since water can facilitate the degradation of some compounds. For example, the addition of water (e.g., 5%) is widely accepted in the pharmaceutical arts as a means of simulating long term storage in order to determine characteristics such as shelf life or the stability of formulations over time. See, e.g., Jens T. Carstensen, Drug Stability: Principles & Practice, 2d. Ed., Marcel Dekker, NY, NY, 1995, pp. 379 80. In effect, water and heat accelerate the decomposition of some compounds. Thus, the effect of water on a formulation can be of great significance since moisture and/or humidity are commonly encountered during manufacture, handling, packaging, storage, shipment, and use of formulations.

Anhydrous pharmaceutical compositions and dosage forms provided herein can be prepared using anhydrous or low moisture containing ingredients and low moisture or low humidity conditions. Pharmaceutical compositions and dosage forms that comprise lactose and at least one active ingredient that comprises a primary or secondary amine can be anhydrous if substantial contact with moisture and/or humidity during manufacturing, packaging, and/or storage is expected.

An anhydrous pharmaceutical composition should be prepared and stored such that its anhydrous nature is maintained. Accordingly, anhydrous compositions can be packaged using materials known to prevent exposure to water such that they can be included in suitable formulary kits. Examples of suitable packaging include, but are not limited to, hermetically sealed foils, plastics, unit dose containers (e.g., vials), blister packs, and strip packs.

Generally, the ingredients of compositions are supplied either separately or mixed together in unit dosage form, for example, as a dry lyophilized powder or water free concentrate in a hermetically sealed container such as an ampoule or sachet indicating the quantity of active agent.

Typical dosage forms comprise a compound provided herein, or a pharmaceutically acceptable salt, solvate or hydrate thereof lie within the range of from about 0.1 mg to about 1000 mg per day, given as a single once-a-day dose in the morning or as divided doses throughout the day taken with food. Particular dosage forms can have about 0.1, 0.2, 0.3, 0.4, 0.5, 1.0, 2.0, 2.5, 5.0, 10.0, 15.0, 20.0, 25.0, 50.0, 100, 200, 250, 500 or 1000 mg of the active compound(s).

Further, the salt forms of L-Ergothioneine as described herein can be administered in a liquid dosage form suitable for oral administration, including, but not limited to a solution or suspension. The salt forms of L-Ergothioneine as described herein can be administered intravenously.

### Topical and Mucosal Delivery

Also provided herein is a method of making a pharmaceutical composition comprising highly purified L-Ergothioneine from crystalline L-Ergothioneine phosphoric acid Form A. In one embodiment, the pharmaceutical composition comprising highly purified L-Ergothioneine is converted to a liquid form. In one embodiment, the liquid form is provided in a dosage form suitable for topical administration as described herein. In one embodiment, the liquid is provided in a dosage form suitable for oral administration, including, but not limited to, a suspension or a solution.

In one embodiment, the pharmaceutical composition comprising highly purified L-Ergothioneine is in a dosage form suitable for topical and mucosal dosage forms, including, but not limited to formulation in products for application to the skin, hair, eyebrows and eyelashes, nails, lips, teeth, and gums. They may be formulated into moisturizers, serums, tonics, gels, creams, sprays, skin balms, lip balms, mouthwashes, teeth strips, sublingual pads, and such. They may be formulated into food or drinks.

Topical and mucosal dosage forms include, but are not limited to, solutions, sprays, aerosols, creams, lotions, ointments, gels, solutions, emulsions, suspensions, or other forms known to one of skill in the art. See, e.g., Remington's Pharmaceutical Sciences, 16^{th}, 18^{th} and 20^{th} eds., Mack Publishing, Easton PA (1980, 1990 & 2000); and Introduction to Pharmaceutical Dosage Forms, 4th ed., Lea & Febiger, Philadelphia (1985). In certain embodiments, the dosage form is selected from creams, lotions, gels, ointments, serum, face and body powder, foundation, color makeup, eyeliner, mascara, antiperspirant, deodorant, and micro-sponge.

The products may be designed to reduce inflammation of the skin, even skin tone, hydrate or moisturize the skin, reduce fine lines and wrinkles, improve skin and such.

Suitable excipients (e.g., carriers and diluents) and other materials that can be used to provide transdermal, topical, and mucosal dosage forms encompassed herein are well known to those skilled in the pharmaceutical arts, and depend on the particular tissue to which a given pharmaceutical composition or dosage form will be applied. With that fact in mind, typical excipients include, but are not limited to, water, acetone, ethanol, ethylene glycol, propylene glycol, butane 1,3 diol, isopropyl myristate, isopropyl palmitate, mineral oil, and mixtures thereof to form lotions, tinctures, creams, emulsions, gels or ointments, which are nontoxic and pharmaceutically acceptable. Moisturizers or humectants can also be added to pharmaceutical compositions and dosage forms if desired. Examples of such additional ingredients are well known in the art. See, e.g., Remington's Pharmaceutical Sciences, 16^{th}, 18^{th} and 20^{th} (eds., Mack Publishing, Easton PA (1980, 1990 & 2000).

Depending on the specific tissue to be treated, additional components may be used prior to, in conjunction with, or subsequent to treatment with active ingredients provided. For example, penetration enhancers can be used to assist in delivering the active ingredients to the tissue. Suitable penetration enhancers include, but are not limited to: acetone; various alcohols such as ethanol, oleyl, and tetrahydrofuryl; alkyl sulfoxides such as dimethyl sulfoxide; dimethyl acetamide; dimethyl formamide; polyethylene glycol; pyrrolidones such as polyvinylpyrrolidone; Kollidon grades (Povidone, Polyvidone); urea; and various water soluble or insoluble sugar esters such as Tween 80 (polysorbate 80) and Span 60 (sorbitan monostearate).

The pH of a pharmaceutical composition or dosage form, or of the tissue to which the pharmaceutical composition or dosage form is applied, may also be adjusted to improve delivery of one or more active ingredients. Similarly, the polarity of a solvent carrier, its ionic strength, or tonicity can be adjusted to improve delivery. Compounds such as stearates can also be added to pharmaceutical compositions or dosage forms to advantageously alter the hydrophilicity or lipophilicity of one or more active ingredients so as to improve delivery. In this regard, stearates can serve as a lipid vehicle for the formulation, as an emulsifying agent or surfactant, and as a delivery enhancing or penetration enhancing agent. Different salts, hydrates or solvates of the active ingredients can be used to further adjust the properties of the resulting composition.

### Methods of Treatment

In one embodiment, a form of L-Ergothioneine provided herein, including the salt forms and crystalline forms thereof and the crystalline L-Ergothioneine Form C, is used to treat a disease or disorder caused by oxidative stress and/or inflammation. Non-limiting examples of disorders, diseases or other conditions associated with oxidative stress include cancer, neurodegenerative disease (Parkinson's disease, Alzheimer's disease, multiple sclerosis and amyolotrophic lateral sclerosis), eye disorders cardiovascular disease, atherosclerosis, sickle cell disease, thrombotic thrombocytopenia purpura, sepsis, cystic fibrosis, chronic fatigue syndrome, kidney disease, diabetes, acute respiratory distress syndrome, gout, arthritis, and other inflammatory diseases. Oxidative stress is involved in several age-related conditions (cardiovascular diseases, chronic obstructive pulmonary disease, chronic kidney disease, neurodegenerative diseases, and cancer), including sarcopenia and frailty. Oxidative stress is also involved in viral infections (HCV infection, HIV infection).

Non-limiting examples of inflammatory diseases or disorders include Alzheimer's, arthritis, asthma, atherosclerosis, Crohn's disease, colitis, cystic fibrosis, dermatitis, diverticulitis, hepatitis, irritable bowel syndrome (IBS), lupus erythematous, muscular dystrophy, nephritis, Parkinson's, rheumatoid arthritis, shingles and ulcerative colitis. Inflammatory diseases also include, for example, stroke, cardiovascular disease, chronic obstructive pulmonary disease (COPD), bronchiectasis, chronic cholecystitis, tuberculosis, Hashimoto's thyroiditis, kidney fibrosis, sepsis, sarcoidosis, silicosis and other pneumoconioses.

### EXAMPLES

### Methods

In various embodiments, XRPD patterns were collected with a PANalytical X′Pert PRO MPD or a PANalytical Empyrean diffractometer using an incident beam of Cu radiation produced using an Optix long, fine-focus source. An elliptically graded multilayer mirror was used to focus Cu Kα X-rays through the specimen and onto the detector. Prior to the analysis, a silicon specimen (NIST SRM 640f) was analyzed to verify that the observed position of the Si 111 peak was consistent with the NIST-certified position. A specimen of the sample was sandwiched between 3-μm-thick films and analyzed in transmission geometry. A beam-stop, short antiscatter extension, and antiscatter knife edge were used to minimize the background generated by air. Soller slits for the incident and diffracted beams were used to minimize broadening from axial divergence. Diffraction patterns were collected using a scanning position-sensitive detector (X′Celerator) located 240 mm from the specimen and Data Collector software v. 5.5. The data acquisition parameters for each pattern are displayed above the appropriate FIG.

In other embodiments, the XRPD patterns were collected with a PANalytical X′Pert PRO MPD diffractometer using an incident beam of Cu Kα radiation produced using a long, fine-focus source and a nickel filter. The diffractometer was configured using the symmetric Bragg-Brentano geometry. Prior to the analysis, a silicon specimen (NIST SRM 640f) was analyzed to verify that the observed position of the Si 111 peak was consistent with the NIST-certified position. A specimen of the sample was prepared as a thin, circular layer centered on a silicon zero-background substrate. Antiscatter slits (SS) were used to minimize the background generated by air. Soller slits for the incident and diffracted beams were used to minimize broadening from axial divergence. Diffraction patterns were collected using a scanning position-sensitive detector (X′Celerator) located 240 mm from the sample and Data Collector software v. 5.5. The data acquisition parameters for each pattern are displayed above the appropriate FIG., including the divergence slit (DS) and the incident-beam SS.

DSC was performed using a Mettler-Toledo DSC3+ differential scanning calorimeter. A tau lag adjustment was performed with indium, tin, and zinc. The temperature and enthalpy were adjusted with octane, phenyl salicylate, indium, tin and zinc. The adjustment was then verified with octane, phenyl salicylate, indium, tin, and zinc. The sample was placed into a hermetically sealed aluminium DSC pan, the weight was accurately recorded, and the sample was inserted into the DSC cell. A weighed aluminium pan configured as the sample pan was placed on the reference side of the cell. The pan lid was pierced prior to sample analysis. The samples were analysed from −25° C. to 250° C. at 10° C./min.

In other embodiments, DSC was performed using a TA Instruments model Q10 differential scanning calorimeter. The instrument was calibrated using indium. The sample was placed into a standard aluminum DSC pan, covered with a lid, and the weight was accurately recorded. An aluminum pan configured as the sample pan was placed on the reference side of the cell. The pan lid was crimped prior to sample analysis. Samples were analyzed in a single run from 25 to 300° C. at a heating rate of 10 or 20° C./min under nitrogen gas.

Thermogravimetric (TG) analyses were performed using a Mettler-Toledo TGA/DSC3+ analyzer. Temperature and enthalpy adjustments were performed using indium, tin, and zinc, and then verified with indium. The balance was verified with calcium oxalate. The sample was placed in an aluminum pan. The pan was hermetically sealed, the lid pierced, and the pan was then inserted into the TG furnace. A weighed aluminum pan configured as the sample pan was placed on the reference platform. The furnace was heated under nitrogen. Samples were analyzed from 25° C. to 350° C. at 10° C./min. Thermogravimetric analyses typically experience a period of equilibration at the start of each analysis, indicated by red parentheses on the thermograms. The starting temperature for relevant weight loss calculations is selected at a point beyond this region (typically above 35° C.) for accuracy. DSC analysis on this instrument is less sensitive than on the DSC3+ differential scanning calorimeter. Therefore, in certain embodiments, samples with sufficient solids were analyzed by both instruments and the TGA thermogram from Mettler-Toledo TGA/DSC3+ analyzer is reported below.

Moisture sorption/desorption data were collected on a VTI SGA-100 Vapor Sorption Analyzer. Samples were not dried prior to analysis. Sorption and desorption data were collected over a range from 5% to 95% RH at 10% RH increments under a dry air purge. The equilibrium criterion used for analysis was less than 0.0100% weight change in 5 minutes with a maximum equilibration time of 3 hours with a 2-minute data logging interval. Data were not corrected for the initial moisture content of the samples.

Ion chromatography analyses were performed using a Dionex ICS-5000⁺ series ion chromatograph. The ICS-5000⁺ consists of two chromatography systems that share an autosampler. The system used for anion detection was equipped with a gradient pump, an eluent generator module, a conductivity detector, and a suppressor (AERS 4 mm). A Dionex UTAC-ULP1 5×23 mm concentrator column was installed in place of the sample loop. A Dionex IonPac™ AG19 4×50 mm guard column and a Dionex IonPac™ AS19 4×250 mm analytical column were installed. Water (18.2 MΩ, dispensed from ELGA Purelab Flex 2) was used to fill the eluent reservoir, for standard preparations, and for autosampler flush. DMSO was used for sample preparation and associated blank injections.

For optical microscopy, samples were observed under a Motic or Wolfe optical microscope with crossed polarizers or under a Leica stereomicroscope with a first order red compensator with crossed polarizers.

The solution NMR spectra were acquired with an Avance 600 MHz spectrometer. The samples were prepared by dissolving approximately 5-10 mg of sample in DMSO-d6 containing TMS.

### Example 1. Salt Studies of L-Ergothioneine

A number of salt forms of L-Ergothioneine were studied and the acidic coformers that were used for the study, in addition to calcium chloride, are presented in Table 1. These experiments generally involved the direct addition of approximately one molar equivalent of the coformer. Samples within this study were protected from light when possible.

Two forms of L-Ergothioneine were previously described in the literature. A crystal form of L-Ergothioneine dihydrate was first reported in Sugihara et al. (Acta Cryst, 1976, B32: 181) and an anhydrate crystalline form of L-Ergothioneine was reported (Hand et al. J. Nat. Prod. 2005, 68:293). Below the anhydrate crystalline form of L-Ergothioneine from Hand et al. is referred to as Form A and the L-Ergothioneine dihydrate from Sugihara et al. is referred to as Form B.

All salt forms discussed below were formed from a mixture of L-Ergothioneine composed predominately of L-Ergothioneine Form A Anhydrate with a minor quality of L-Ergothioneine Form B Dihydrate (referred to as L-Ergothioneine Form A/B below).

TABLE 1
Coformers used in Salt Studies

| Wermuth Class | pKa1 | pKa2 | pKa3 | |
|---|---|---|---|---|
| 1 | 4.76 | | | Acetic Acid |
| | 4.44 | 5.44 | | Adipic |
| | 1.88 | 3.65 | 9.6 | L-Aspartic |
| | 3.13 | 4.76 | 6.40 | Citric |
| | 3.03 | 4.38 | — | Fumaric |
| | ~−6.1 | — | — | Hydrochloric |
| | 1.92 | 6.23 | — | Maleic |
| | 1.96 | 7.12 | 12.32 | Phosphoric |
| | −3 | 1.92 | — | Sulfuric |
| | 3.02 | 4.36 | — | (+)-L-Tartaric |
| 2 | 4.19 | — | — | Benzoic |
| | 2.17 | — | — | (+)-Camphor-10- |
| | | | | sulfonic |
| | −1.2 | — | — | Methanesulfonic |
| GRAS | — | — | — | Calcium chloride |

Unique crystalline materials were isolated from the acetate, benzoate, fumarate, hydrochloride, maleate, phosphate, sulfate, and tartrate salt/cocrystal study and from the ionic calcium chloride cocrystal study. The experimental conditions and characterization of the crystalline materials are discussed below.

The approximate solubility of the L-Ergothioneine Form A/B mixture in various solvents was determined (Table 2). Overall, poor solubility was observed in the organic solvents tested at both ambient temperature and 60° C.

TABLE 2
Solubility of L-Ergothioneine mixture used in salt studies

| Solvent | Temperature | Solubility (mg/mL)* |
|---|---|---|
| Acetone | RT | <3 |
| 1,4-dioxane | RT | <3 |
| Et₂O | RT | <2 |
| EtOAc | RT | <3 |
| HFIPA | RT | 3 |
| MeOH | RT | <2 |
| | 60° C. | <2 |
| THF | RT | <21 |

*Solubilities are calculated based on the total solvent used to give a solution; actual solubilities may be greater because of the volume of the solvent portions used or a slow rate dissolution. Values are rounded to the nearest whole number. If dissolution did not occur as determined by visual assessment, the value is reported as “<”. If dissolution occurred as determined by the visual assessment after the addition of the first aliquot, the value is reported as “>”.

### L-Ergothioneine Phosphoric Acid Cocrystal Form A

L-Ergothioneine phosphoric acid cocrystal Form A is anhydrous. The cocrystal was generated by heating a slurry of L-Ergothioneine Form A/B in acetic acid to 75° C. and adding water before adding a molar equivalent of phosphoric acid. The heat was turned off and the solution was allowed to stand at room temperature overnight protected from light to afford opaque white solids with no birefringence upon observation by polarized light microscopy. A summary of this synthetic procedure and a second procedure that resulted in L-Ergothioneine phosphoric acid cocrystal Form A and free form anhydrous L-Ergothioneine is presented in Table 3.

TABLE 3
Studies for the Isolation of L-Ergothioneine
Phosphoric Acid Cocrystal Form A

| | Technique | Result |
|---|---|---|
| 1 | 1. L-Ergothioneine Form A/B contacted with | Phosphate A + |
| | a molar eq. of acid in MeOH | free form |
| | 2. slurry, RT, 5 d protected from light | anhydrous |
| | 3. filtered, N₂ dried | |
| 2 | 1. L-Ergothioneine Form A/B in acetic acid, | Phosphate A |
| | 75° C. hot plate | |
| | 2. water added | |
| | 3. molar eq. of acid added, heat off | |
| | 4. RT, overnight protected from light | |
| | 5. filtered, N₂ dried | |
| 3 | Phosphate Form A from (2) above filtrate | Phosphate A |
| | 1. fast evaporation partial | |
| | 2. filtered, N₂ dried | |

B = birefringence and NB = no birefringence upon observation by polarized light microscopy.

A crystal of sufficient size and quality for single crystal structure elucidation was culled. The crystal system was monoclinic and the space group was C2. The cell parameters and calculated volume were: a=30.7749 (5) Å, b=6.01160 (10) Å, c=7.80400 (10) Å, α=90°, β=95.7830 (10)°, γ=90°, V=1436.44 (4) Å3. The formula weight was 327.29 μg mol⁻¹ with Z=4, resulting in a calculated density\\\ of 1.513 g cm⁻³. Additional details of the crystal data and crystallographic data collection parameters are summarized in Table 4.

TABLE 4
Crystal Data and Data Collection Parameters for
L-Ergothioneine Phosphoric Acid Cocrystal Form A

| | |
|---|---|
| Empirical formula | C₉H₁₈N₃O₆PS |
| Formula weight (g mol⁻¹) | 327.29 |
| Temperature (K) | 299.77(10) |
| Wavelength (Å) | 1.54184 |
| Crystal system | monoclinic |
| Space group | C2 |
| Unit cell parameters | |
| a = 30.7749(5) Å | a = 90° |
| b = 6.01160(10) Å | /3 = 95.7830(10)° |
| c = 7.80400(10) Å | γ = 90° |
| Unit cell volume (Å³) | 1436.44(4) |
| Cell formula units, Z | 4 |
| Calculated density (g cm⁻³) | 1.513 |
| Absorption coefficient (mm⁻¹) | 3.345 |
| F(000) | 688 |
| Crystal size (mm³) | 0.11 × 0.08 × 0.04 |
| Reflections used for cell measurement | 5366 |
| O range for cell measurement | 5.6890°-75.5940° |
| Total reflections collected | 6274 |
| Index ranges | −37 ≤ h ≤ 38; −7 ≤ |
| | k ≤ 7; −9 ≤ l < 8 |
| O range for data collection | Omin = 5.698°, |
| | Omax = 75.789° |
| Completeness to Omax | 99% |
| Completeness to Ofull = 67.684° | 99.9% |
| Absorption correction | multi-scan |
| Transmission coefficient range | 0.838-1.000 |
| Refinement method | full matrix least-squares on F2 |
| Independent reflections | 2886 [Rint = 0.0159, |
| | Rσ = 0.0206] |
| Reflections [I > 2σ(I)] | 2834 |
| Reflections/restraints/parameters | 2886/1/253 |
| Goodness-of-fit on _{F}2 | S = 1.04 |
| Final residuals [I > 2σ(I)] | R = 0.0212, Rw = 0.0561 |
| Final residuals [all reflections] | R = 0.0216, Rw = 0.0564 |
| Largest diff. peak and hole (e Å⁻³) | 0.167, −0.190 |
| Max/mean shift/standard uncertainty | 0.002/0.000 |
| Absolute structure determination | Flack parameter: 0.000(7) |

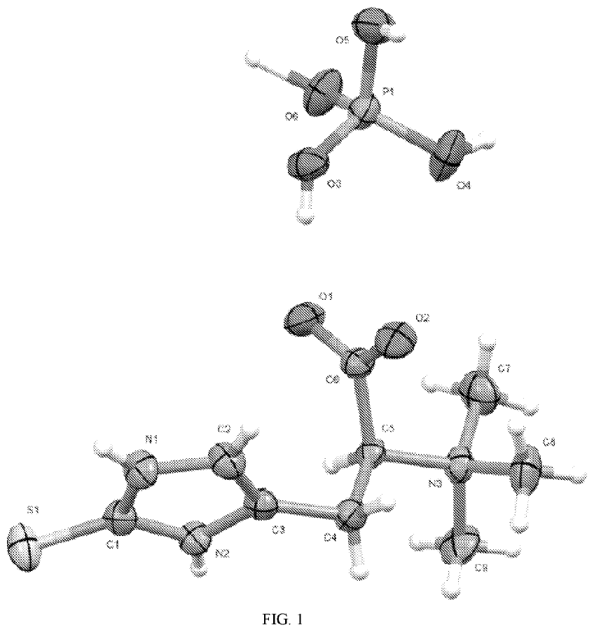

An atomic displacement ellipsoid drawing of L-Ergothioneine phosphoric acid cocrystal is shown in FIG. 1. The asymmetric unit shown contains one L-Ergothioneine molecule and one phosphoric acid molecule. Four hydrogens are depicted on the acid, however, two of them reside on a 2-fold axis and the other half of those two hydrogen atoms is symmetry generated. Therefore, the acid stoichiometry is H₃PO₄ and the form is a co-crystal. The calculated powder pattern is and displayed in FIG. 2.

Table 5 is a list of the XRPD peaks for the L-Ergothioneine phosphoric acid cocrystal Form A.

TABLE 5
Peaks of the XRPD pattern of L-Ergothioneine
Phosphoric Acid Cocrystal Form A

| °2θ | d space (Å) | Intensity (%) |
|---|---|---|
| 5.77 ± 0.20 | 15.312 ± 0.550 | 4 |
| 11.38 ± 0.20* | 7.768 ± 0.138 | 52 |
| 11.57 ± 0.20 | 7.645 ± 0.134 | 3 |
| 12.25 ± 0.20 | 7.219 ± 0.119 | 8 |
| 13.27 ± 0.20 | 6.666 ± 0.102 | 7 |
| 14.99 ± 0.20* | 5.905 ± 0.079 | 100 |
| 15.41 ± 0.20* | 5.746 ± 0.075 | 13 |
| 17.10 ± 0.20 | 5.182 ± 0.061 | 52 |
| 17.36 ± 0.20 | 5.103 ± 0.059 | 3 |
| 18.68 ± 0.20* | 4.745 ± 0.051 | 87 |
| 19.05 ± 0.20* | 4.654 ± 0.049 | 27 |
| 19.82 ± 0.20* | 4.476 ± 0.045 | 29 |
| 20.09 ± 0.20 | 4.417 ± 0.044 | 6 |
| 20.69 ± 0.20* | 4.290 ± 0.041 | 68 |
| 21.07 ± 0.20* | 4.212 ± 0.040 | 71 |
| 21.78 ± 0.20* | 4.078 ± 0.037 | 53 |
| 22.93 ± 0.20 | 3.875 ± 0.034 | 26 |
| 23.03 ± 0.20 | 3.859 ± 0.033 | 30 |
| 23.25 ± 0.20 | 3.823 ± 0.033 | 4 |
| 24.20 ± 0.20 | 3.675 ± 0.030 | 14 |
| 24.38 ± 0.20* | 3.648 ± 0.030 | 48 |
| 24.63 ± 0.20* | 3.611 ± 0.029 | 13 |
| 24.88 ± 0.20 | 3.575 ± 0.029 | 3 |
| 25.17 ± 0.20* | 3.535 ± 0.028 | 12 |
| 26.82 ± 0.20 | 3.321 ± 0.024 | 19 |
| 27.22 ± 0.20* | 3.273 ± 0.024 | 23 |
| 27.42 ± 0.20 | 3.250 ± 0.023 | 13 |
| 27.73 ± 0.20* | 3.215 ± 0.023 | 15 |
| 27.98 ± 0.20 | 3.187 ± 0.022 | 9 |
| 28.54 ± 0.20* | 3.125 ± 0.022 | 15 |
| 29.16 ± 0.20 | 3.060 ± 0.021 | 4 |
| 29.43 ± 0.20 | 3.033 ± 0.020 | 3 |
| 29.68 ± 0.20* | 3.007 ± 0.020 | 22 |
| 29.91 ± 0.20 | 2.984 ± 0.020 | 4 |
| 30.27 ± 0.20 | 2.951 ± 0.019 | 35 |

*Prominent peaks

Solution ¹H NMR spectrum was consistent with the chemical structure of L-Ergothioneine and contained peaks attributed to residual crystallization solvent that integrate to 0.01 mol/mol acetic acid. The TGA (FIG. 3) exhibited a 2.4% weight loss (up to 117° C.) coinciding with multiple volatilization endotherms in the DSC (FIG. 4). The final endotherm with an onset of 234° C. is likely from concomitant melt and decomposition. Based on the known anhydrous crystal structure, the weight loss is likely due to volatilization of moisture and suggests that the cocrystal is hygroscopic.

### L-Ergothioneine Acetate Form A

L-Ergothioneine acetate Form A was obtained by stirring a slurry of 159.3 mg of L-Ergothioneine Form A/B in 10 mL glacial acetic acid at 400 RPM for 6 days at ambient temperature, protected from light. Solids were collected by water aspirated vacuum filtration.

The XRPD pattern of acetate Form A is shown in FIG. 5. The form has a monoclinic unit cell likely containing two hydrogen L-Ergothioneine cations and two acetate anions. Consequently, the formula unit volume of 356 Å3 calculated from the indexing results would be consistent with an unsolvated monoacetate salt (Hofmann, D W M. Fast Estimation of Crystal Densities. Acta. Cryst. 2002; B57 (3-2): 489-93). Table 6 is a list of the XRPD peaks for the L-Ergothioneine Acetate Form A.

TABLE 6
Peaks of the XRPD pattern of L-Ergothioneine Acetate Form A

| °2θ | d space (Å) | Intensity (%) |
|---|---|---|
| 5.30 ± 0.20* | 16.663 ± 0.653 | 41 |
| 10.63 ± 0.20* | 8.316 ± 0.159 | 11 |
| 12.52 ± 0.20* | 7.065 ± 0.114 | 50 |
| 14.51 ± 0.20 | 6.101 ± 0.085 | 5 |
| 14.82 ± 0.20* | 5.971 ± 0.081 | 11 |
| 15.83 ± 0.20* | 5.595 ± 0.071 | 26 |
| 15.98 ± 0.20 | 5.542 ± 0.070 | 6 |
| 17.93 ± 0.20* | 4.942 ± 0.055 | 15 |
| 18.35 ± 0.20* | 4.831 ± 0.053 | 35 |
| 19.50 ± 0.20* | 4.548 ± 0.047 | 100 |
| 20.84 ± 0.20* | 4.259 ± 0.041 | 33 |
| 21.06 ± 0.20* | 4.215 ± 0.040 | 18 |
| 21.34 ± 0.20 | 4.160 ± 0.039 | 9 |
| 21.91 ± 0.20* | 4.053 ± 0.037 | 61 |
| 22.16 ± 0.20* | 4.008 ± 0.036 | 48 |
| 22.65 ± 0.20 | 3.923 ± 0.035 | 9 |
| 23.40 ± 0.20* | 3.799 ± 0.032 | 63 |
| 23.71 ± 0.20* | 3.749 ± 0.031 | 35 |
| 24.73 ± 0.20* | 3.597 ± 0.029 | 17 |
| 25.17 ± 0.20 | 3.535 ± 0.028 | 7 |
| 25.44 ± 0.20 | 3.499 ± 0.027 | 7 |
| 26.12 ± 0.20 | 3.409 ± 0.026 | 6 |
| 26.81 ± 0.20 | 3.323 ± 0.025 | 13 |
| 27.22 ± 0.20 | 3.273 ± 0.024 | 24 |
| 27.37 ± 0.20 | 3.255 ± 0.024 | 9 |
| 29.00 ± 0.20* | 3.077 ± 0.021 | 28 |
| 29.35 ± 0.20 | 3.041 ± 0.020 | 5 |
| 29.63 ± 0.20 | 3.012 ± 0.020 | 7 |
| 30.05 ± 0.20* | 2.971 ± 0.019 | 12 |
| 30.53 ± 0.20* | 2.925 ± 0.019 | 14 |

*Prominent peaks

The solution ¹H NMR spectrum was consistent with the chemical structure of L-Ergothioneine and contained peaks attributed to acetic acid.

The thermograms are provided in FIG. 6 and FIG. 7. The TGA (FIG. 6) showed 33% weight loss up to 148° C. and an additional 14% weight loss up to 239° C. As shown in FIG. 7, a broad DSC endotherm, concurrent with the first volatilization step in the TGA, exhibited a peak maximum of 106° C. The second endotherm, concurrent with the second volatilization step, exhibited an onset of 180° C. The third endotherm with an onset of 259° C. is likely from the decomposition of L-Ergothioneine. Since the second weight loss step is consistent with ˜1 mol/mol acetic acid, the first weight loss step is likely due to loss of residual solvent and/or moisture.

### L-Ergothioneine Benzoate Form A

L-Ergothioneine benzoate Form A was obtained by heating a solution of 54.4 mg of L-Ergothioneine Form A/B and 28.6 mg of benzoic acid in 1.5 mL of 67:33 v/v water/MeOH. Nucleation was forced from solution by removing it from the heat source and allowing it to cool to ambient temperature. The sample was then refrigerated for 3 days followed by 1 day in a freezer prior to recovering solids by water aspirated vacuum filtration.

The XRPD pattern of L-Ergothioneine benzoate Form A as shown in FIG. 8. The form has a monoclinic unit cell likely containing two hydrogen L-Ergothioneine cations and two benzoate anions. Consequently, the formula unit volume of 442 Å3 calculated from the indexing results would be consistent with an anhydrous monobenzoate salt (Hofmann, D W M. Fast Estimation of Crystal Densities. Acta. Cryst. 2002; B57 (3-2): 489-93). Table 7 is a list of the XRPD peaks for the L-Ergothioneine benzoate Form A.

TABLE 7
Peaks of the XRPD pattern of L-Ergothioneine Benzoate Form A

| °2θ | d space (Å) | Intensity (%) |
|---|---|---|
| 4.20 ± 0.20* | 21.040 ± 1.053 | 100 |
| 8.41 ± 0.20* | 10.508 ± 0.256 | 8 |
| 12.55 ± 0.20* | 7.046 ± 0.114 | 35 |
| 12.80 ± 0.20 | 6.909 ± 0.109 | 4 |
| 13.67 ± 0.20* | 6.472 ± 0.096 | 14 |
| 14.34 ± 0.20* | 6.172 ± 0.087 | 13 |
| 15.48 ± 0.20* | 5.721 ± 0.074 | 21 |
| 16.85 ± 0.20 | 5.259 ± 0.063 | 14 |
| 17.13 ± 0.20* | 5.172 ± 0.061 | 9 |
| 18.80 ± 0.20* | 4.716 ± 0.050 | 29 |
| 19.57 ± 0.20 | 4.532 ± 0.046 | 50 |
| 19.67 ± 0.20 | 4.509 ± 0.046 | 45 |
| 19.95 ± 0.20* | 4.446 ± 0.045 | 22 |
| 20.29 ± 0.20 | 4.373 ± 0.043 | 3 |
| 20.74 ± 0.20* | 4.279 ± 0.041 | 14 |
| 21.14 ± 0.20 | 4.199 ± 0.040 | 2 |
| 21.83 ± 0.20 | 4.069 ± 0.037 | 2 |
| 22.21 ± 0.20 | 3.999 ± 0.036 | 2 |
| 22.56 ± 0.20* | 3.938 ± 0.035 | 59 |
| 23.41 ± 0.20* | 3.796 ± 0.032 | 21 |
| 24.08 ± 0.20* | 3.692 ± 0.030 | 26 |
| 24.99 ± 0.20 | 3.561 ± 0.028 | 10 |
| 25.39 ± 0.20 | 3.506 ± 0.027 | 4 |
| 25.84 ± 0.20 | 3.445 ± 0.026 | 7 |
| 25.94 ± 0.20 | 3.432 ± 0.026 | 9 |
| 26.10 ± 0.20 | 3.411 ± 0.026 | 4 |
| 26.82 ± 0.20 | 3.321 ± 0.024 | 6 |
| 27.06 ± 0.20 | 3.293 ± 0.024 | 2 |
| 27.58 ± 0.20* | 3.232 ± 0.023 | 10 |
| 27.88 ± 0.20* | 3.198 ± 0.023 | 12 |
| 29.38 ± 0.20 | 3.038 ± 0.020 | 9 |

*Prominent peaks

The solution ¹H NMR spectrum was consistent with the chemical structure of L-Ergothioneine and contained peaks attributed to benzoic acid. As shown in FIG. 9, negligible weight loss by TGA (0.4% upon heating up to 209° C.) is consistent with an anhydrate. The single endotherm with an onset of 238° C. observed in the DSC (FIG. 10) is likely from concomitant melt and decomposition.

### L-Ergothioneine Calcium Chloride Material A

L-Ergothioneine calcium chloride Material A was obtained by heating a slurry of 173.9 mg of L-Ergothioneine Form A/B in 1 mL of water to 65° C. A faint turbid solution was afforded with the addition of 110.5 mg of calcium chloride to the slurry. The sample was refrigerated for 3 days and became clear. The clear solution was evaporated to dryness under nitrogen. The resulting material appeared to deliquesce upon removal from the nitrogen stream. The material was tacky and appeared to deliquesce.

The XRPD pattern representing L-Ergothioneine calcium chloride Material A is provided in FIG. 11. Table 8 is a list of the XRPD peaks for the L-Ergothioneine calcium chloride Form A.

TABLE 8
Peaks of the XRPD pattern of L-Ergothioneine
Calcium Chloride Material A

| °2θ | d space (Å) | Intensity (%) |
|---|---|---|
| 5.22 ± 0.20 | 16.930 ± 0.675 | 14 |
| 10.46 ± 0.20 | 8.448 ± 0.164 | 14 |
| 12.07 ± 0.20* | 7.328 ± 0.123 | 51 |
| 14.41 ± 0.20 | 6.143 ± 0.086 | 21 |
| 15.09 ± 0.20* | 5.866 ± 0.078 | 74 |
| 15.61 ± 0.20 | 5.672 ± 0.073 | 28 |
| 17.30 ± 0.20 | 5.122 ± 0.059 | 20 |
| 17.83 ± 0.20* | 4.970 ± 0.056 | 46 |
| 18.65 ± 0.20 | 4.754 ± 0.051 | 44 |
| 18.80 ± 0.20 | 4.716 ± 0.050 | 100 |
| 19.32 ± 0.20 | 4.591 ± 0.048 | 25 |
| 19.85 ± 0.20 | 4.468 ± 0.045 | 24 |
| 20.26 ± 0.20 | 4.381 ± 0.043 | 25 |
| 21.01 ± 0.20 | 4.225 ± 0.040 | 29 |
| 21.19 ± 0.20* | 4.189 ± 0.039 | 56 |
| 21.39 ± 0.20 | 4.150 ± 0.039 | 25 |
| 22.04 ± 0.20* | 4.029 ± 0.036 | 84 |
| 22.83 ± 0.20 | 3.892 ± 0.034 | 24 |
| 23.06 ± 0.20 | 3.853 ± 0.033 | 31 |
| 23.23 ± 0.20* | 3.826 ± 0.033 | 45 |
| 23.68 ± 0.20 | 3.754 ± 0.032 | 25 |
| 24.08 ± 0.20 | 3.692 ± 0.030 | 22 |
| 24.30 ± 0.20 | 3.660 ± 0.030 | 26 |
| 24.43 ± 0.20* | 3.640 ± 0.030 | 42 |
| 25.39 ± 0.20 | 3.506 ± 0.027 | 20 |
| 26.14 ± 0.20 | 3.407 ± 0.026 | 27 |
| 26.47 ± 0.20 | 3.364 ± 0.025 | 26 |
| 26.62 ± 0.20 | 3.346 ± 0.025 | 32 |
| 27.66 ± 0.20 | 3.223 ± 0.023 | 33 |
| 28.03 ± 0.20 | 3.181 ± 0.022 | 26 |
| 28.31 ± 0.20 | 3.150 ± 0.022 | 34 |
| 28.51 ± 0.20 | 3.128 ± 0.022 | 28 |
| 29.65 ± 0.20 | 3.011 ± 0.020 | 27 |

*Prominent peaks

### L-Ergothioneine Hemifumarate Form A

L-Ergothioneine hemifumarate Form A was obtained by stirring a slurry of 180.9 mg of L-Ergothioneine Form A/B and 91.8 mg of fumaric acid in 10 ml of methanol at 400 RPM for 6 days at ambient temperature protected from light. Solids were collected by water aspirated vacuum filtration and characterized.

The XRPD pattern of hemifumarate Form A is shown in FIG. 12. The form has a monoclinic unit cell likely containing two hydrogen L-Ergothioneine cations and one fumarate anion. Consequently, the formula unit volume of 337 Å3 calculated from the indexing results would be consistent with an anhydrous hemifumarate salt (Hofmann, D W M. Fast Estimation of Crystal Densities. Acta. Cryst. 2002; B57 (3-2): 489-93). Table 9 is a list of the XRPD peaks for the L-Ergothioneine hemifumarate Form A.

TABLE 9
Peaks of the XRPD pattern of L-Ergothioneine
hemifumarate Form A

| °2θ | d space (Å) | Intensity (%) |
|---|---|---|
| 5.65 ± 0.20* | 15.629 ± 0.573 | 37 |
| 11.32 ± 0.20* | 7.814 ± 0.140 | 11 |
| 12.18 ± 0.20* | 7.258 ± 0.121 | 32 |
| 12.57 ± 0.20 | 7.037 ± 0.113 | 9 |
| 14.27 ± 0.20* | 6.200 ± 0.088 | 38 |
| 15.09 ± 0.20 | 5.866 ± 0.078 | 3 |
| 15.26 ± 0.20 | 5.802 ± 0.077 | 3 |
| 16.13 ± 0.20* | 5.491 ± 0.068 | 53 |
| 17.01 ± 0.20 | 5.207 ± 0.061 | 4 |
| 17.98 ± 0.20* | 4.929 ± 0.055 | 65 |
| 18.90 ± 0.20* | 4.691 ± 0.050 | 17 |
| 19.27 ± 0.20* | 4.602 ± 0.048 | 100 |
| 19.45 ± 0.20 | 4.559 ± 0.047 | 25 |
| 19.69 ± 0.20* | 4.506 ± 0.046 | 34 |
| 20.82 ± 0.20 | 4.262 ± 0.041 | 8 |
| 21.51 ± 0.20* | 4.128 ± 0.038 | 76 |
| 22.56 ± 0.20 | 3.938 ± 0.035 | 9 |
| 22.83 ± 0.20* | 3.892 ± 0.034 | 99 |
| 23.56 ± 0.20* | 3.772 ± 0.032 | 24 |
| 24.03 ± 0.20* | 3.700 ± 0.031 | 43 |
| 24.57 ± 0.20* | 3.621 ± 0.029 | 60 |
| 25.35 ± 0.20 | 3.510 ± 0.027 | 3 |
| 26.12 ± 0.20* | 3.409 ± 0.026 | 27 |
| 27.24 ± 0.20 | 3.271 ± 0.024 | 11 |
| 27.39 ± 0.20 | 3.253 ± 0.023 | 14 |
| 27.64 ± 0.20 | 3.224 ± 0.023 | 13 |
| 28.51 ± 0.20 | 3.128 ± 0.022 | 52 |
| 28.69 ± 0.20 | 3.109 ± 0.021 | 16 |
| 28.93 ± 0.20 | 3.084 ± 0.021 | 15 |
| 29.61 ± 0.20 | 3.014 ± 0.020 | 4 |

*Prominent peaks

The solution 1H NMR spectrum was consistent with the chemical structure of L-Ergothioneine and contains peaks attributed to fumaric acid that integrate to 0.5 mol/mol of acid. Negligible weight loss by TGA (0.4% upon heating up to 224° C.) is consistent with an anhydrate (FIG. 13). The single endotherm with an onset of 244° C. observed in the DSC is likely from concomitant melt and decomposition (FIG. 14).

### L-Ergothioneine Hydrochloride Form A and Mixtures

L-Ergothioneine hydrochloride Form A was isolated alone and in mixtures with a minor quantity of HCl Material B with HCl Material C. Experimental conditions for each are summarized in Table 10. Form A was isolated starting from the mixture of Form A HCl and Form C HCl.

TABLE 10
Studies for the Isolation of L-Ergothioneine Hydrochloride Form A

| Technique | Result |
|---|---|
| 1. L-Ergothioneine Form A/B in a MeOH slurry | HCl C + |
| 2. a molar eq. acid added slowly | HCl A |
| 3. filtered | |
| 4. refrigerated, 4 d | |
| 5. fast evaporation partial | |
| 6. evaporation | |
| complete | |
| 1. L-Ergothioneine Form A/B in aMeOH slurry | HCl A + |
| 2. a molar eq. acid added | HCl B |
| 3. MeOH added | |
| 4. filtered | |
| 5. EtOAc added | |
| 6. 8609-44-01 seed added with EtOAc | |
| 7. refrigerated, 30 min | |
| 8. freezer, 1 d | |
| 9. filtered, N₂ dried | |
| HCl C + HCl A filtrate | HCl A |
| 1. transferred to vial | |
| 2. N₂ evaporation | |
| partial | |
| 3. EtOAc added | |
| 4. MeOH added | |
| 5. filtered, EtOAc | |
| rinse, N₂ dried | |

The XRPD pattern of HCl Form A is shown in FIG. 15. The form has a monoclinic unit cell likely containing two hydrogen L-Ergothioneine cations and two chlorine anions. Consequently, the formula unit volume of 358 Å3 calculated from the indexing results would be consistent with a monochloride that can theoretically also accommodate up to two mol/mol of water (Hofmann, D W M. Fast Estimation of Crystal Densities. Acta. Cryst. 2002; B57 (3-2): 489-93).

Table 11 is a list of the XRPD peaks for the L-Ergothioneine HCl Form A.

TABLE 11
Peaks of the XRPD pattern of L-Ergothioneine
Hydrochloride Form A

| °2θ | d space (Å) | Intensity (%) |
|---|---|---|
| 12.10 ± 0.20* | 7.306 ± 0.122 | 11 |
| 13.39 ± 0.20* | 6.607 ± 0.100 | 25 |
| 14.03 ± 0.20 | 6.309 ± 0.091 | 18 |
| 14.13 ± 0.20* | 6.264 ± 0.090 | 52 |
| 17.22 ± 0.20* | 5.146 ± 0.060 | 29 |
| 17.52 ± 0.20* | 5.058 ± 0.058 | 64 |
| 18.17 ± 0.20* | 4.878 ± 0.054 | 13 |
| 18.64 ± 0.20 | 4.757 ± 0.051 | 12 |
| 18.91 ± 0.20* | 4.690 ± 0.050 | 100 |
| 21.16 ± 0.20* | 4.195 ± 0.040 | 56 |
| 22.58 ± 0.20 | 3.934 ± 0.035 | 6 |
| 22.85 ± 0.20 | 3.889 ± 0.034 | 5 |
| 23.92 ± 0.20 | 3.717 ± 0.031 | 6 |
| 24.17 ± 0.20* | 3.679 ± 0.030 | 39 |
| 24.34 ± 0.20 | 3.654 ± 0.030 | 8 |
| 24.52 ± 0.20* | 3.627 ± 0.029 | 24 |
| 24.69 ± 0.20 | 3.603 ± 0.029 | 8 |
| 25.37 ± 0.20* | 3.507 ± 0.027 | 62 |
| 25.71 ± 0.20 | 3.463 ± 0.027 | 22 |
| 26.53 ± 0.20 | 3.358 ± 0.025 | 11 |
| 26.71 ± 0.20 | 3.335 ± 0.025 | 8 |
| 26.94 ± 0.20 | 3.306 ± 0.024 | 10 |
| 27.11 ± 0.20 | 3.286 ± 0.024 | 5 |
| 27.68 ⊥ 0.20 | 3.220 ⊥ 0.023 | 7 |
| 28.06 ± 0.20 | 3.177 ± 0.022 | 10 |
| 28.25 ± 0.20 | 3.157 ± 0.022 | 10 |
| 28.57 ± 0.20 | 3.122 ± 0.022 | 19 |
| 28.97 ± 0.20* | 3.080 ± 0.021 | 36 |
| 29.48 ± 0.20 | 3.027 ± 0.020 | 12 |

*Prominent peaks

The solution ¹H NMR spectrum of the mixture substantially composed of L-Ergothioneine Hydrochloride Form A and a minor quantity of HCl Material B was consistent with the chemical structure of L-Ergothioneine and contained peaks attributed to EtOAc that integrate to 0.01 mol/mol. Ion chromatography (IC) analysis of the mixture was consistent with a monochloride salt.

Thermograms of the mixture substantially composed of L-Ergothioneine Hydrochloride Form A and a minor quantity of HCl Material B is shown in FIG. 16 and FIG. 17. The TGA thermogram (FIG. 16) exhibited a 6.3% weight loss (up to 158° C.) concurrent with a volatilization endotherm in the DSC (FIG. 17) near 120° C. Since negligible residual organic solvent was evident by NMR (discussed above), it can be inferred that the weight loss is due to the volatilization of approximately 1 mol/mol water. Since the sample is substantially composed of L-Ergothioneine Hydrochloride Form A, it can be reasoned that the water loss is attributed to that form. Therefore, L-Ergothioneine Hydrochloride Form A is likely a monohydrate. The final endotherm with an onset at 201° C. is likely the concomitant melt/decomposition of the resulting dihydrate.

### L-Ergothioneine Hemimaleate Form A and Maleate Material B Mixture

A mixture of L-Ergothioneine Hemimaleate Form A and Maleate Material B was isolated by heating a slurry of 158.1 mg of L-Ergothioneine Form A/B in 3 mL of methanol to 65° C. Complete dissolution was provided with the addition of 80.1 mg maleic acid in 0.3 mL water. The solution was allowed to cool to ambient temperature and then refrigerated for 3 days. The precipitants were collected by water aspirated vacuum filtration and dried briefly under nitrogen.

The predominant component in the XRPD pattern exhibited by the mixture was visually similar to hemifumarate Form A (FIG. 12). Based on the visual similarity, it can be inferred that the predominant component is isostructural to hemifumarate Form A, and was designated hemimaleate Form A. The remaining unknown peaks were designated as maleate Material B. Since the material was a mixture, no further characterization was obtained.

### L-Ergothioneine Sulfate Form A

A solution of 30.8 μL sulfuric acid and 129.2 mg of L-Ergothioneine Form A/B in 7.7 mL of a 91:09 v/v glacial acetic acid/water was generated at 75° C. The solution was left to cool to ambient temperature and then evaporated under nitrogen (protected from light) to dryness to afford L-Ergothioneine sulfate Form A.

The XRPD pattern of sulfate Form A is shown in FIG. 18. The form has an orthorhombic unit cell likely containing two hydrogen L-Ergothioneine cations and two sulfate anions. Consequently, the formula unit volume of 360 Å3 calculated from the indexing results would be consistent with an unsolvated monosulfate salt (Hofmann, D W M. Fast Estimation of Crystal Densities. Acta. Cryst. 2002; B57 (3-2): 489-93). IC analysis for the sulfate anion is also consistent with a monosulfate salt.

Table 12 is a list of the XRPD peaks for the L-Ergothioneine sulfate Form A.

TABLE 12
Peaks of the XRPD pattern of L-Ergothioneine Sulfate Form A

| °2θ | d space (Å) | Intensity (%) |
|---|---|---|
| 5.83 ± 0.20* | 15.137 ± 0.537 | 10 |
| 11.97 ± 0.20* | 7.390 ± 0.125 | 10 |
| 13.02 ± 0.20 | 6.794 ± 0.106 | 3 |
| 14.52 ± 0.20* | 6.094 ± 0.085 | 29 |
| 16.71 ± 0.20* | 5.300 ± 0.064 | 72 |
| 17.55 ± 0.20 | 5.050 ± 0.058 | 5 |
| 18.42 ± 0.20 | 4.813 ± 0.052 | 22 |
| 18.60 ± 0.20* | 4.766 ± 0.051 | 76 |
| 19.29 ± 0.20 | 4.598 ± 0.048 | 7 |
| 20.41 ± 0.20* | 4.349 ± 0.043 | 100 |
| 21.09 ± 0.20* | 4.209 ± 0.040 | 94 |
| 21.83 ± 0.20* | 4.069 ± 0.037 | 63 |
| 22.65 ± 0.20* | 3.923 ± 0.035 | 16 |
| 23.33 ± 0.20 | 3.810 ± 0.032 | 19 |
| 23.55 ± 0.20 | 3.775 ± 0.032 | 57 |
| 24.07 ± 0.20 | 3.695 ± 0.031 | 7 |
| 24.95 ± 0.20 | 3.566 ± 0.028 | 11 |
| 25.05 ± 0.20 | 3.552 ± 0.028 | 10 |
| 25.50 ± 0.20 | 3.490 ± 0.027 | 5 |
| 26.15 ± 0.20 | 3.404 ± 0.026 | 9 |
| 27.66 ± 0.20 | 3.223 ± 0.023 | 20 |
| 28.04 ± 0.20* | 3.179 ± 0.022 | 25 |
| 28.64 ± 0.20 | 3.114 ± 0.021 | 13 |
| 29.01 ± 0.20 | 3.075 ± 0.021 | 6 |
| 29.50 ± 0.20 | 3.026 ± 0.020 | 7 |
| 29.98 ± 0.20 | 2.978 ± 0.020 | 17 |

*Prominent peaks

The TGA thermogram (FIG. 19) exhibited a 4.5% weight loss up to 187° C. Thermal decomposition (FIG. 20), which caused the material to expand out of the pan, was evident above this temperature. The weight loss is likely due to volatilization of moisture and suggests that the monosulfate is hygroscopic.

### L-Ergothioneine Hemitartrate Form A

A slurry of 182.3 mg of L-Ergothioneine Form A/B and 118.1 mg L-tartaric acid was generated in 5 mL of methanol and stirred at 400 RPM for 6 days at ambient, protected from light. Solids were collected by water aspirated vacuum filtration to afford L-Ergothioneine hemitartrate Form A.

The XRPD pattern of hemitartrate Form A is shown as FIG. 21. The form has a triclinic unit cell likely containing two hydrogen L-Ergothioneine cations and one tartrate anion. Consequently, the formula unit volume of 353 Å3 calculated from the indexing results would be consistent with an unsolvated hemitartrate salt (Hofmann, D W M. Fast Estimation of Crystal Densities. Acta. Cryst. 2002; B57 (3-2): 489-93).

Table 13 is a list of the XRPD peaks for the L-Ergothioneine hemitartrate Form A.

TABLE 13
Peaks of the XRPD pattern of L-Ergothioneine
Hemitartrate Form A

| °2θ | d space (Å) | Intensity (%) |
|---|---|---|
| 5.55 ± 0.20* | 15.911 ± 0.594 | 20 |
| 11.13 ± 0.20 | 7.942 ± 0.145 | 9 |
| 12.08 ± 0.20 | 7.318 ± 0.123 | 51 |
| 12.22 ± 0.20 | 7.238 ± 0.120 | 15 |
| 14.32 ± 0.20* | 6.179 ± 0.087 | 65 |
| 14.64 ± 0.20 | 6.045 ± 0.083 | 9 |
| 14.97 ± 0.20 | 5.911 ± 0.080 | 13 |
| 15.53 ± 0.20* | 5.703 ± 0.074 | 31 |
| 16.41 ± 0.20* | 5.397 ± 0.066 | 32 |
| 18.10 ± 0.20* | 4.897 ± 0.054 | 90 |
| 18.53 ± 0.20* | 4.783 ± 0.052 | 100 |
| 18.85 ⊥ 0.20* | 4.703 ⊥ 0.050 | 29 |
| 19.42 ± 0.20 | 4.567 ± 0.047 | 83 |
| 19.69 ± 0.20 | 4.506 ± 0.046 | 14 |
| 20.07 ± 0.20 | 4.420 ± 0.044 | 12 |
| 20.69 ± 0.20* | 4.290 ± 0.041 | 26 |
| 21.33 ± 0.20 | 4.163 ± 0.039 | 47 |
| 21.56 ± 0.20 | 4.119 ± 0.038 | 83 |
| 22.65 ± 0.20 | 3.923 ± 0.035 | 22 |
| 23.18 ± 0.20 | 3.834 ± 0.033 | 91 |
| 23.77 ± 0.20 | 3.741 ± 0.031 | 16 |
| 24.50 ± 0.20 | 3.630 ± 0.029 | 46 |
| 26.10 ± 0.20 | 3.411 ± 0.026 | 58 |
| 26.56 ± 0.20* | 3.354 ± 0.025 | 18 |
| 26.92 ± 0.20* | 3.309 ± 0.024 | 23 |
| 27.69 ± 0.20 | 3.219 ± 0.023 | 20 |
| 28.09 ± 0.20 | 3.174 ± 0.022 | 30 |
| 28.46 ± 0.20 | 3.134 ± 0.022 | 28 |
| 28.88 ± 0.20 | 3.089 ± 0.021 | 14 |
| 29.18 ± 0.20 | 3.058 ± 0.021 | 12 |
| 29.56 ± 0.20 | 3.019 ± 0.020 | 12 |

*Prominent peaks

The material was difficult to filter and was isolated as a wet cake; the residual methanol is likely due to the inefficiency of the filtration process. The 1H NMR spectrum is consistent with the chemical structure of L-Ergothioneine and contains peaks attributed to tartaric acid and methanol that integrate to 0.5 and 2.8 mol/mol, respectively. The TGA thermogram (FIG. 22) exhibits a 15% weight loss (up to 138° C.) coinciding with volatilization endotherm near 56° C. in the DSC (FIG. 23), likely from the volatilization of residual methanol. The final endotherm with an onset of 235° C. is likely from concomitant melt and decomposition.

### Example 2. Characterization of L-Ergothioneine Form C Anhydrate

L-Ergothioneine Form C Anhydrate was identified by single crystal X-ray diffraction.

The crystal system was orthorhombic and the space group was P2₁2₁2₁. The cell parameters and calculated volume were: a=8.9959 (2) Å, b=10.8401 (2) Å, c=11.5979 (3) Å, α=90°, β=90°, γ=90°, V=1130.99 (4) Å³. The molecular weight was 229.30 g mol⁻¹ with Z=4, resulting in a calculated density of 1.347 g cm⁻³. Further details of the crystal data and crystallographic data collection parameters are summarized in Table 14.

TABLE 14
Crystal Data and Data Collection Parameters
for L-Ergothioneine Form C Anhydrate

| | |
|---|---|
| Empirical formula | C₉H₁₅N₃O₂S |
| Formula weight (g mol⁻¹) | 229.30 |
| Temperature (K) | 299.58(11) |
| Wavelength (Å) | 1.54184 |
| Crystal system | orthorhombic |
| Space group | P212121 |
| Unit cell parameters | |
| a = 8.9959(2) Å | a = 90° |
| b = 10.8401(2) Å | /1 = 90° |
| c = 11.5979(3) Å | y = 90° |
| Unit cell volume (Å³) | 1130.99(4) |
| Cell formula units, Z | 4 |
| Calculated density (g cm⁻³) | 1.347 |
| Absorption coefficient (mm⁻¹) | 2.446 |
| F(000) | 488 |
| Crystal size (mm³) | 0.1 × 0.07 × 0.06 |
| Reflections used for cell measurement | 3337 |
| O range for cell measurement | 5.5750°-75.4900° |
| Total reflections collected | 4844 |
| Index ranges | −11 ≤ h ≤ 7; −13 ≤ k ≤ 13; −12 ≤ l ≤ 14 |
| O range for data collection | Omin = 5.586°, Omax = 75.820° |
| Completeness to Omax | 97.7% |
| Completeness to Ofull = 67.684° | 100% |
| Absorption correction | multi-scan |
| Transmission coefficient range | 0.707-1.000 |
| Refinement method | full matrix least-squares on F2 |
| Independent reflections | 2268 [Rint = 0.0360, Rσ = 0.0422] |
| Reflections [I > 2σ(I)] | 2179 |
| Reflections/restraints/parameters | 2268/0/196 |
| Goodness-of-fit on _{F}2 | S = 1.06 |
| Final residuals [I > 2σ(I)] | R = 0.0411, Rw = 0.1037 |
| Final residuals [all reflections] | R = 0.0423, Rw = 0.1047 |
| Largest diff. peak and hole (e Å⁻³) | 0.486, −0.204 |
| Max/mean shift/standard uncertainty | 0.000/0.000 |
| Absolute structure determination | Flack parameter: −0.024(14) |

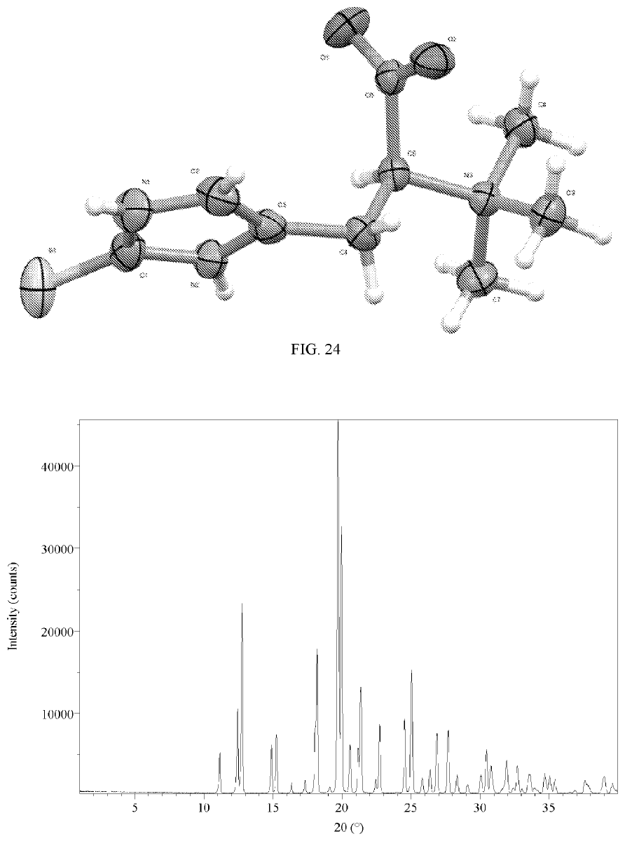

An atomic displacement ellipsoid drawing of L-Ergothioneine is shown in FIG. 24. The asymmetric unit shown contains one L-Ergothioneine molecule. The calculated powder pattern is shown in FIG. 25 and L-Ergothioneine Form C was characterized by an XRPD pattern comprising peaks in Table 15.

TABLE 15
Peaks of the XRPD pattern of L-Ergothioneine Form C Anhydrate

| °2θ | d space (Å) | Intensity (%) |
|---|---|---|
| 11.17 ± 0.20* | 7.916 ± 0.144 | 12 |
| 12.44 ± 0.20* | 7.110 ± 0.116 | 23 |
| 12.77 ± 0.20* | 6.925 ± 0.110 | 51 |
| 14.90 ± 0.20* | 5.943 ± 0.080 | 14 |
| 15.26 ± 0.20* | 5.800 ± 0.077 | 16 |
| 16.35 ± 0.20 | 5.417 ± 0.067 | 3 |
| 17.32 ± 0.20 | 5.116 ± 0.059 | 4 |
| 18.05 ± 0.20 | 4.910 ± 0.055 | 18 |
| 18.19 ± 0.20 | 4.874 ± 0.054 | 39 |
| 19.09 ± 0.20 | 4.645 ± 0.049 | 3 |
| 19.72 ± 0.20* | 4.497 ± 0.046 | 100 |
| 19.96 ± 0.20* | 4.445 ± 0.045 | 72 |
| 20.59 ± 0.20* | 4.309 ± 0.042 | 14 |
| 21.16 ± 0.20* | 4.195 ± 0.040 | 13 |
| 21.36 ± 0.20* | 4.156 ± 0.039 | 29 |
| 22.45 ± 0.20 | 3.957 ± 0.035 | 5 |
| 22.72 ± 0.20* | 3.911 ± 0.034 | 19 |
| 24.55 ± 0.20 | 3.622 ± 0.029 | 21 |
| 25.04 ± 0.20* | 3.553 ± 0.028 | 34 |
| 25.82 ± 0.20 | 3.447 ± 0.026 | 5 |
| 26.39 ± 0.20 | 3.374 ± 0.025 | 7 |
| 26.88 ± 0.20* | 3.314 ± 0.024 | 16 |
| 27.70 ± 0.20* | 3.218 ± 0.023 | 18 |
| 28.35 ± 0.20 | 3.146 ± 0.022 | 6 |
| 29.12 ± 0.20 | 3.064 ± 0.021 | 3 |
| 30.07 ± 0.20 | 2.969 ± 0.019 | 6 |
| 30.47 ± 0.20* | 2.931 ± 0.019 | 12 |

*Prominent peaks

Two other forms of L-Ergothioneine, designated as L-Ergothioneine Form A Anhydrate and L-Ergothioneine Form B Dihydrate when described herein, are known. FIG. 26 are representative XRPD patterns of the known L-Ergothioneine Form A Anhydrate, the known Form B Dihydrate, and the Form C Anhydrate.

The TGA and DSC thermograms for Form C Anhydrate are presented in FIG. 27 and FIG. 28, respectively. A negligible TGA weight loss of 0.3% was observed up to 250° C., consistent with an anhydrate. The endotherm observed in the DSC with an onset of 265° C. is likely the melt with concomitant decomposition.

### Example 3. Physical Stability of L-Ergothioneine Form A Anhydrate, L-Ergothioneine Form B Dihydrate, and L-Ergothioneine Form C Anhydrate

### L-Ergothioneine Form A Anhydrate

The DVS isotherm indicated that Form A exhibits low hygroscopicity from 5 to 85% RH and significant hygroscopicity above 85% RH. Consistent with the DVS result, the critical water activity between the anhydrate and the dihydrate was tentatively determined to fall between 0.76 and 0.85 aw, above which hydration to Form B Dihydrate will occur. This is also consistent with the physical stability assessment at 33, 75, and 95% RH for 22 days; Form A Anhydrate remained Form A below the critical water activity, while hydration to Form B Dihydrate occurred above (Table 16).

TABLE 16
Physical Stability L-Ergothioneine Form A Anhydrate

| Condition | Results |
|---|---|
| 33% RH, RT, 22 days | A |
| 75% RH, RT, 22 days | A |
| 95% RH, RT, 22 days | B |

### L-Ergothioneine Form B Dihydrate

The physical stability assessment at 33, 75, and 95% RH for 22 days showed that Form B Dihydrate was sustained at 75% RH and above within the timeframe evaluated. The material did partially dehydrate to a small quantity of both Forms A and C Anhydrates at 33% RH.

### L-Ergothioneine Form C Anhydrate

Form C Anhydrate is monotropically related and thermodynamically metastable relative to Form A. Form C exhibits a congruent melt and decomposition onset near 265° C. as described in Example 2.

The DVS isotherm indicated that Form C exhibits limited hygroscopicity from 5 to 85% RH and significant hygroscopicity above 85% RH (FIG. 29). The material lost 0.3 wt % upon equilibration at 5% RH. During the sorption cycle, the material gained approximately 0.9 wt % up through 85% RH. At 95% RH, more than 10.9 wt % gain was observed (equivalent to more than 1.5 mol/mol water gain). Equilibrium weight was achieved at each RH sorption step except at 95% RH, indicating that additional weight gain may be possible if sustained at that condition for longer. On the desorption cycle, the material lost 5.5 wt % until reaching 75% RH and then remained at a stable weight plateau until reaching 45% RH, providing significant hysteresis. While equilibrium weight was not achieved at several of the desorption steps below 45% RH, complete desorption equivalent to the loss of 1 mol/mol of water was eventually achieved once reaching 5% RH. The recovered material was identified as a mixture of Form C Anhydrate, Form B Dihydrate, and another form by XRPD.

The physical stability assessment at 75 and 95% RH for 22 days is consistent the DVS results above; Form C remained at 75% RH, while hydration to Form B Dihydrate occurred at 95% RH (Table 17).

TABLE 17
Physical Stability for L-Ergothioneine Form C Anhydrate

| Condition | Results |
|---|---|
| 75% RH, RT, 22 days | Form C |
| 95% RH, RT, 22 days | Form B |

### Example 4. Thermodynamic Stability

Phase transitions of solids can be thermodynamically reversible or irreversible. Crystalline forms which transform reversibly at a specific transition temperature are called enantiotropic polymorphs. If the crystalline forms are not interconvertible under these conditions, the system is monotropic (one thermodynamically stable form). Several rules help predict the relative thermodynamic stability of polymorphs and whether the relationship between the polymorphs is enantiotropic or monotropic. The density and heat of fusion rules, justified on a statistical mechanical basis, are used here for guidance of monotropy or enantiotropy.

The density rule, which is based on Kitaǐgorodskiǐ's principle of closest packing for molecular crystals (Kitaĭgorodskiĭ, A. I. Molecular Crystals and Molecules; Academic Press: New York, 1973), states that, for a non-hydrogen-bonded system at absolute zero, the most stable polymorph will have the highest density, because of stronger intermolecular van der Waals interactions. Thus, according to this rule, the crystal structure with the most efficient packing will also have the lowest free energy. This assumes that hydrogen bonding (long range effect) is not a major parameter in crystal packing. The densities determined from the indexing results of Form A (1.42 g cm⁻³) and the single crystal structure of Form C (1.35 g cm⁻³) suggest that, at absolute zero, Form A is more stable than Form C.

The melt onsets and heats of fusion, obtained from calorimetry data, are normally useful to estimate the relative physical stabilities of polymorphic pairs. Due to concomitant decomposition at the melt, the heats of fusion values for each form of L-Ergothioneine from DSC thermograms are unsuitable for this purpose. Although the difference between melt onsets is not statistically significant, the melt onset of Form C (265.3° C.) is slightly lower than that of Form A (265.6° C.). Because the densest form (above) also exhibits the higher melting temperature, Form A is consistent as the more thermodynamically stable form from a monotropically related pair.

Interconversion slurry experiments are a solution-mediated process that provides a pathway for the less soluble (more stable) crystal to grow at the expense of the more soluble crystal form (Bernstein, J. Polymorphism in Molecular Crystals. Clarendon Press, Oxford, 2006; Polymorphism in Pharmaceutical Solids. Brittain, Harry G. ed. Marcek Dekker, Inc. New York. 1999). Outside the formation of a solvate or degradation, the resulting more stable polymorph from an interconversion experiment is independent of the solvent used because the more thermodynamically stable polymorph has a lower energy and therefore lower solubility. The choice of solvent affects the kinetics of polymorph conversion and not the thermodynamic relationship between polymorphic forms (Gu, CH., Young, V. Jr., Grant, DJ. J. Pharm. Sci 2001; 90 (11): 1878-1890). Competitive slurry experiments conducted in solvent systems that provide at least about 3 mg/mL (or about 8 mM) solubility should complete within a reasonable timeframe.

Finding a solvent system with adequate solubility that did not form solvates with L-Ergothioneine was not possible. Slurry interconversion experiments in ethanol (at 75° C.) and methanol (at room temperature) were still attempted to confirm the thermodynamic relationship between Forms A and C (Table 18).

TABLE 18
Forms A and C Interconversion Experiments
at Room Temperature and 75° C.

| Solvent | Condition | Result |
|---|---|---|
| EtOH | 75° C., 14 days | A + C |
| EtOH | 75° C., 28 days | A + C (minor) |
| MeOH | RT, 14 days | A |

Although the dihydrate of L-Ergothioneine can form in the presence of water, aqueous methanol mixtures were utilized to enhance solubility. To avoid the dihydrate, the aqueous mixtures were prepared below the critical water activity of its formation. Form C was triturated in the aqueous mixtures and monitored for conversion to Form A by XRPD after 5 days (Table 19). Conversion to Form A within that timeframe was observed in all experiments (20:80 through 10:90 water/methanol) except for the experiment conducted at the lowest volume fraction of water and solubility.

TABLE 19
Trituration of Form C in Aqueous Methanol below
the Critical Water Activity at Room Temperature

| v/v Solvent(s) | a_{w} | Condition | Result |
|---|---|---|---|
| 20:80 water/MeOH | 0.47 | RT, 5 d | A |
| 15:85 water/MeOH | 0.39 | RT, 5 d | A |
| 10:90 water/MeOH | 0.29 | RT, 5 d | A + C (minor) |
| 3:97 water/MeOH | 0.10 | RT, 5 d | C |

The interconversion and trituration experiments confirm that Form A is the more thermodynamically stable form, relative to Form C, at the temperatures evaluated.

### Example 5. Stability of the Hydrates

Water activity is related to relative humidity in that RH %=a_{w}×100. Therefore, it is possible to directly relate the stability of an anhydrous/hydrate system in slurry experiments to solid state stability. Literature suggests that the slurry technique at controlled water activities provides an accurate method of rapidly predicting the physically stable form in anhydrous/hydrate systems (Ticehurst M D, Storey R A, Claire W. Int J Pharm. 2002; 247:1-10; Sacchetti M. Int J Pharm. 2004; 273:195-202; Zhu H, Yuen C, Grant D J W. 1. Theophylline. Int J Pharm. 1996; 135:151-160; Zhu H, Grant D J W. 2. Ampicillin. Int J Pharm. 1996; 139:33-43).

The method is particularly valuable when slow kinetics of conversion in the solid state prevents reaching true equilibrium in a reasonable timeframe, since solvent-mediated transformation accelerates the conversion process.

The effect of relative humidity (RH) and water activity (aw) on the hydration state of L-Ergothioneine was investigated through both static exposure to different relative humidity (Table 17 and Table 18) and competitive water activity trituration experiments at room temperature (Table 20). The resulting solids were characterized by XRPD. These experiments were used to establish the relative humidity ranges for form stability of the thermodynamically stable anhydrate Form A and Form B Dihydrate. The critical water activity between the anhydrate and the dihydrate falls between 0.76 and 0.85 a_{w}. This range is consistent with the DVS isotherms for both Forms A and C Anhydrate.

TABLE 20
Water Activity Experiments of Form A and Form C at RT

| v/v Solvent(s) | a_{w} | Method | Observation | Result |
|---|---|---|---|---|
| 70:30 water/MeOH | 0.86 | RT, 13 d | Birefringent plates | B (PO) |
| 50:50 water/MeOH | 0.76 | RT, 13 d | Birefringent plates, wet solids | A |
| | | | | A + B + anotherform |
| | | | | A + B + anotherform |
| | | | | A + B + anotherform |
| | | | | A + B + anotherform |
| | | | | A + B + anotherform |
| 20:80 water/MeOH | 0.47 | RT, 8 d | Small birefringent blades | B + A + C |
| 20:80 water/MeOH | 0.47 | RT, 14 d | Unknown morphology, birefringent | A + B |
| 15:85 water/MeOH | 0.39 | RT, 8 d | Small birefringent blades | B + A + C + anotherform |
| 15:85 water/MeOH | 0.39 | RT, 14 d | Unknown morphology, birefringent | A |

### Example 6. Characterization of DL-Ergothioneine Form A

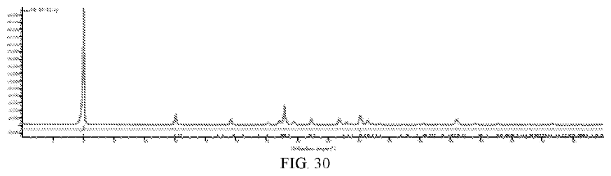

FIG. 30 is the XRPD pattern of DL-Ergothioneine Form A, which shows a mixture predominantly composed of DL-Ergothioneine Form A with a minor phase impurity of an unknown form. The peaks associated with the phase impurities were ignored. DL-Ergothioneine Form A was successfully indexed from the remaining peaks as a single unit cell. The form is in a non-chiral monoclinic space group, consistent with a racemate. The unit cell contains four DL-Ergothioneine molecules. Consequently, the formula unit volume of 330 Å³ calculated from the indexing results would be consistent with a hydrate that can theoretically accommodate up to 2 mol/mol of water (Coelho, A. A., J. Appl. Cryst. 36 (2003) 86-95). The peaks associated with the minor phase impurity are not consistent with any known forms of L-Ergothioneine.

TABLE 21
Crystal Data and Data Collection Parameters
for DL-Ergothioneine Form A

| | | | |
|---|---|---|---|
| Space group | P2_{1/C} | Cell Volume (Å³) | 1319.51 |
| a (Å) | 15.103 | α (°) | 90 |
| b (Å) | 6.059 | β (°) | 77.12 |
| c (Å) | 14.791 | γ (°) | 90 |
| dₛₐₘₚ (mm) | −0.094 | | |
| R_{wp} | 9.1% * | | |

* many minor peaks are not accounted for by this indexing solution, indicating the presence of an impurity: 12.5°, 15.0°, 15.8°, 18.0°, 21.2°, 25.6°, 27.9°, 29.8°, . . .

Molecular volumes are predicted according to Hofmann [0]: DL-Ergothioneine, C₉H₁₅N₂O₂S: 284.41 Å³, P2₁/_{c}, predicts a total volume of 4×284.41=1137.64 Å³. Void volume, dv=1319.51−1137.64=181.87 Å³.

The embodiments and examples described above are intended to be merely illustrative and non-limiting. Those skilled in the art will recognize or will be able to ascertain using no more than routine experimentation, numerous equivalents of specific compounds, materials and procedures. All such equivalents are considered to be within the scope and are encompassed by the appended claims.

What is claimed is:

1. An L-Ergothioneine salt selected from the group consisting of L-Ergothioneine acetate, L-Ergothioneine benzoate, L-Ergothioneine hemifumurate, L-Ergothioneine phosphoric acid, L-Ergothioneine sulfate, L-Ergothioneine hemitartrate, L Ergothioneine hemimaleate, L-Ergothioneine maleate, L-Ergothioneine adipic acid, L Ergothioneine L-aspartic acid, L-Ergothioneine citric acid, L-Ergothioneine camphorsulfonic acid, and L-Ergothioneine methanesulfonic sulfonic acid.

2. L-Ergothioneine phosphoric acid of the formula:

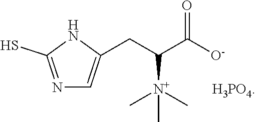

3. The L-Ergothioneine phosphoric acid of claim 1, wherein the L-Ergothioneine phosphoric acid is crystalline.

4. The crystalline L-Ergothioneine phosphoric acid of claim 3, wherein the L-Ergothioneine phosphoric acid is a co-crystal.

5. Co-crystal L-Ergothioneine phosphoric acid (Form A) characterized by an X-ray powder diffraction (XRPD) comprising peaks at 14.99, 18.68, and 21.07±0.2° 2θ using Cu Kα radiation.

6. The co-crystal of claim 5, characterized by an X-ray powder diffraction (XRPD) comprising peaks at 14.99, 18.68, 20.69, and 21.07±0.2° 2θ.

7. The co-crystal of claim 6, characterized by an XRPD comprising peaks at 11.38, 14.99, 18.68, 20.69, 21.07, and 21.78±0.2° 2θ.

8. The co-crystal of claim 7, characterized by an XRPD comprising peaks at 11.38, 14.99, 15.41, 18.68, 19.05, 19.82, 20.69, 21.07, 21.78, 24.38, 24.63, 25.17, 27.22, 27.73, 28.54, and 29.68±0.2° 2θ.

9. The co-crystal of any one of claims 5-8, characterized by a differential scanning calorimetry (DSC) thermograph having two endotherms wherein the first has an endotherm maximum between about 75° C. and 85° C. and wherein the second an endotherm maximum between about 235° C. and 245° C.

10. The co-crystal of claim 9, wherein characterized by a DSC thermograph having two endotherms wherein the first endotherm a maximum at about 77° C. and wherein the second endotherm a maximum at about 241° C.

11. The co-crystal of any one of claims 5-8, characterized by a DSC thermograph substantially similar to that set forth in FIG. 4.

12. The co-crystal of any one of claims 5-11, characterized by a weight loss in the range of about 1% and 5% when heated up to about 120° C. in a thermogravimetric analysis (TGA).

13. The co-crystal of claim 12, characterized by a weight loss of about 2.4% when heated up to about 117° C. in a TGA.

14. The co-crystal of any one of claims 5-11, characterized by TGA substantially similar to that set forth in FIG. 3.

15. A pharmaceutical composition comprising the L-Ergothioneine phosphoric acid of any one of claims 2-4 or the co-crystal L-Ergothioneine phosphoric acid (Form A) of any one of claims 5-14 in a pharmaceutically acceptable carrier.

16. A pharmaceutical composition comprising L-Ergothioneine prepared from the co-crystal L-Ergothioneine phosphoric acid of any one of claims 5-14 in a pharmaceutically acceptable carrier.

17. The pharmaceutical composition of claim 15-16, in a dosage form suitable for topical administration.

18. A method for the purification of L-Ergothioneine comprising the step of contacting L-Ergothioneine with a sufficient amount of phosphoric acid under conditions suitable to form L-Ergothioneine phosphoric acid salt.

19. The method of claim 18, further comprising mixing the L-Ergothioneine in a protic solvent and, optionally water, prior to or concomitant with the contacting step.

20. The method of claim 19, wherein the protic solvent is selected from the group consisting of methanol, ethanol, isopropanol, formic acid, acetic acid, and combinations thereof.

21. A crystalline form of L-Ergothioneine anhydrate (Form C) characterized by an X-ray powder diffraction (XRPD) comprising peaks at 11.17 and 12.77±0.2° 2θ using Cu Kα radiation.

22. The crystalline form of claim 21, characterized by an XRPD comprising peaks at 11.17, 12.44, 12.77, 19.72, 19.96, and 22.72±0.2° 2θ.

23. A crystalline form of L-Ergothioneine anhydrate (Form C) characterized by an XRPD comprising peaks at 12.44, 12.77, 19.72, 19.96, 21.36, and 25.04±0.2° 2θ.

24. The crystalline form of claim 23, characterized by an XRPD comprising peaks at 11.17, 12.44, 12.77, 14.90, 15.26, 19.72, 19.96, 20.59, 21.16, 21.36, 22.72, 25.04, 26.88, 27.70, and 30.47±0.2° 2θ.

25. The crystalline form of any one of claims 21-23, characterized by a differential scanning calorimetry (DSC) thermograph having an endotherm with an onset between about 260° C. and 270° C.

26. The crystalline form of claim 25, characterized by a differential scanning calorimetry (DSC) thermograph having an endotherm with an onset of about 265° C.

27. The crystalline form of any one of claims 21-23, characterized by a DSC thermograph substantially similar to that set forth in FIG. 28.

28. The crystalline form of any one of claims 21-27, characterized by a weight loss in the range of 0% to about 1% when heated up to about 255° C. in a thermogravimetric analysis (TGA).

29. The crystalline form of claim 28, characterized by a weight loss of about 0.3% when heated up to about 250° C. in a TGA.

30. The crystalline form of any one of claims 21-27, characterized by TGA substantially similar to that set forth in FIG. 27.

31. A crystalline form of L-Ergothioneine acetate (Form A) characterized by an X-ray powder diffraction (XRPD) comprising peaks at 5.30, 12.52, 19.50, 20.84, 21.91, 22.16, 23.40, and 23.71±0.2° 2θ using Cu Kα radiation.

32. The crystalline form of claim 31, characterized by an XRPD comprising peaks at 5.30, 10.63, 12.52, 14.82, 15.83, 17.93, 18.35, 19.50, 20.84, 21.06, 21.91, 22.16, 23.40, 23.71, 24.73, 29.00, 30.05, and 30.53±0.2° 2θ.

33. A crystalline form of L-Ergothioneine benzoate (Form A) characterized by an X-ray powder diffraction (XRPD) comprising peaks at 4.20, 12.55, 18.80, 19.95, 22.56, and 24.08±0.2° 2θ using Cu Kα radiation.

34. The crystalline form of claim 33, characterized by an XRPD comprising peaks at 4.20, 8.41, 12.55, 13.67, 14.34, 15.48, 17.13, 18.80, 19.95, 20.74, 22.56, 23.41, 24.08, 27.58, and 27.88±0.2° 2θ.

35. A crystalline form of L-Ergothioneine hemifumarate (Form A) characterized by an X-ray powder diffraction (XRPD) comprising peaks at 16.13, 17.98, 19.27, 21.51, 22.83, and 24.57±0.2° 2θ using Cu Kα radiation.

36. The crystalline form of claim 35, characterized by an XRPD comprising peaks at 5.65, 11.32, 12.18, 14.27, 16.13, 17.98, 18.90, 19.27, 19.69, 21.51, 22.83, 23.56, 24.03, 24.57, and 26.12±0.2° 2θ.

37. A crystalline form of L-Ergothioneine hydrochloride (Form A) characterized by an X-ray powder diffraction (XRPD) comprising peaks at 14.13, 17.52, 18.91, 21.16, 24.17, and 25.37±0.2° 2θ using Cu Kα radiation.

38. The crystalline form of claim 37, characterized by an XRPD comprising peaks at 12.10, 13.39, 14.13, 17.22, 17.52, 18.17, 18.91, 21.16, 24.17, 24.52, 25.37, and 28.97±0.2° 2θ.

39. A crystalline form of L-Ergothioneine sulfate (Form A) characterized by an X-ray powder diffraction (XRPD) comprising peaks at 16.71, 18.60, 20.41, and 21.09 5.83, 11.97, 14.52, 16.71, 18.60, 20.41, 21.09, 21.83, 22.65, and 28.04±0.2° 2θ using Cu Kα radiation.

40. The crystalline form of claim 39, characterized by an XRPD comprising peaks at 5.83, 11.97, 14.52, 16.71, 18.60, 20.41, 21.09, 21.83, 22.65, and 28.04±0.2° 2θ.

41. A crystalline form of L-Ergothioneine hemitartrate (Form A) characterized by an X-ray powder diffraction (XRPD) comprising peaks at 14.32, 15.53, 16.41, 18.10, and 18.53±0.2° 2θ using Cu Kα radiation.

42. The crystalline form of claim 41, characterized by an XRPD comprising peaks at 5.55, 14.32, 15.53, 16.41, 18.10, 18.53, 18.85, 20.69, 26.56, and 26.92±0.2° 2θ.

43. A crystalline form of DL-Ergothioneine (Form A) characterized by an XRPD pattern substantially similar to that set forth in FIG. 30.

44. A pharmaceutical composition comprising the crystalline form of any one of claims 21-43 in a pharmaceutically acceptable carrier.

45. A method for the treatment of a disease or disorder caused by oxidative stress and/or inflammation comprising administering the crystalline form of any one of claims 21-43 or a pharmaceutical composition of claim 44.
